(19) United States
(12) Patent Application Publication
(10) Pub. No.: US 2004/0044028 A1
(43) Pub. Date: Mar. 4, 2004

(54) Combinations of omega-3 fatty acids and cyclooxygenase-2 inhibitors for treatment or prevention of cardiovascular disease and treatment or prevention of cancer

(76) Inventor: Mark G. Obukowicz, Kirkwood, MO (US)

Correspondence Address: Pharmacia Corporation, Corporate Patent Department, 800 N. Lindbergh Blvd., Mail Zone O4E, St. Louis, MO, 63167, US

(21) Appl. No.: 10/113,269

(22) Filed: Apr. 1, 2002

Related U.S. Application Data
(60) Provisional application No. 60/280,183, filed on Mar. 30, 2001.

(51) Int. Cl. A61K 31/4745; A61K 31/415; A61K 31/365; A61K 31/201

(52) U.S. Cl. 514/303; 514/406; 514/471; 514/560

(57) ABSTRACT

Combinations of omega-3 polyunsaturated fatty acids (PUFAS) and cyclooxygenase-2 selective inhibitors for treatment or prevention of cardiovascular disease, inflammation related disorders or cancer are disclosed. The preferred omega-3 PUFAs of the present invention have from between eighteen and twenty-two carbon atoms, and more preferably from twenty to twenty-two carbon atoms.

### BACKGROUND OF THE INVENTION

1. Field of the Invention

The present invention relates to treatment and prevention of disease, and more particularly to novel combinations for the treatment and prevention of cardiovascular disease, inflamation, diabetes, and cancer, particularly epithelial cell cancers such as colon cancer, lung cancer, breast cancer, and prostate cancer.

2. Cardiovascular Benefits of Omega-3 Polyunsaturated Fatty Acids

Cardiovascular disease is the number one cause of mortality in the world. Many cardiac disorders (eg, coronary artery disease [CAD], systemic hypertension, bicuspid aortic valve, hypertrophic cardiomyopathy, mitral valve prolapse) have a heritable basis. Although the precise pathogenesis of CAD is unclear, the risk factors are well known: high blood levels of low density lipoprotein cholesterol (LDL-C) and lipoprotein a, low blood levels of high density lipoprotein cholesterol (HDL-C) and serum vitamin E, and poor physical fitness. High blood levels of triglycerides and insulin reflecting insulin resistance may be risk factors, but the data are less clear. CAD risk is increased by tobacco use; diets high in fat and calories and low in phytochemicals (found in fruits and vegetables), fiber, and vitamins E and C, or diets with relatively low levels of omega-3 polyunsaturated fatty acids (PUFAs); poor stress management; and inactivity. Several systemic diseases (e.g., hypertension, diabetes, hypothyroidism) are also associated with increased CAD risk.

In the elderly, decreased baroreceptor responsiveness, coupled with decreased arterial compliance, accounts for frequent orthostatic hypotension. Some NSAIDs may cause renal sodium (Na⁺) retention and inhibit prostaglandin-induced vasodilation.

Most subjects using selective cyclooxygenase-2 (COX-2) inhibiting drugs have osteoarthritis, and the vast majority of those are elderly. Being elderly, these subjects are at a greater risk for cardiovascular disease.

Compelling data showing the cardiovascular-protective benefits of omega-3 polyunsaturated fatty acids (PUFAs) have accumulated over the past 30 years. Epidemiological studies supporting the cardiovascular-protective benefits of omega-3 PUFAs include primary studies, for example Burchfiel C M, Reed D M, Strong J P, Sharp D S, Chyou P -O and Rodriguez B L (1996). Predictors of myocardial lesions in men with minimal coronary atherosclerosis at autopsy. (The Honolulu Heart Program.) Ann Epidemiol 6: 137-146.

Another such primary study by Kromhout D, Bosschieter E B and De Lezenne Coulander C (1985) entitled “The inverse relation between fish consumption and 20-year mortality from coronary heart disease.” N Engl J Med 312: 1205-1209.

Finally, a third such study by Albert C M, Hennekens C H, O'Donnell C J, Ajani U A, Carey V J, Willett W C, Ruskin J N and Manson J E (1998) entitled “Fish consumption and risk of sudden cardiac death.” JAMA 279: 23-28. showed an inverse correlation between increased intake of omega-3 PUFAs and sudden death from myocardial infarction.

Secondary studies have been reported by Burr M L, Fehily A M, Gilbert J F, Rogers S, Holliday R M, Sweetnam P M, Elwood P C and Deadman N M (1989) in “Effects of changes in fat, fish, and fibre intakes on death and myocardial reinfarction: Diet and Reinfarction Trial (DART)” The Lancet 2: 757-761.

Another secondary study by de Lorgeril M, Salen P, Martin J L, et al. entitled “Mediterranean diet, traditional risk factors, and the rate of cardiovascular complications after myocardial infarction: final report of the Lyon Diet Heart Study” Circulation (1999;99:779-785) demonstrated decreased cardiovascular disorder events when subjects were fed a diet which was, among other things, high in omega-3 polyunsaturated fatty acids.

More recently, results from the first intervention trial (GISSI) with omega-3 PUFAs were published in The Lancet (1999, 354:447-455). Survivors of a primary myocardial infarction were administered placebo, supplements of an omega-3 PUFA (purified EPA/DHA combination), vitamin E, or omega-3 PUFA+vitamin E for three and one-half years. Efficacy was only observed in the omega-3 PUFA groups; a significant decrease in all causes of mortality occurred due to reductions in coronary heart disease and coronary vascular disease mortality. Total mortality was reduced by 20% and sudden death by 45% in the patients randomized to 850 mg of n-3 PUFAs per day.

The cytokines interleukin-1 and tumor necrosis factor contribute to the pathogenesis of inflammatory diseases, and potentially, atherogenesis. It is known that oral supplementation of omega-3 fatty acids to diets of healthy volunteers suppresses the capacity of mononuclear cells to synthesize interleukin-1 and tumor necrosis factor. However, the mechanism by which omega-3 fatty acids exert suppression of cytokine synthesis remains unknown.

3. Anti-inflammatory Benefits of Omega-3 Polyunsaturated Fatty Acids

Omega-3 PUFAs have been shown to inhibit inflamation through suppression of monocyte production of interleukin-1 (IL-1β and IL-1α) and tumor necrosis factor. Endres et al. (1989) New England Journal of Medicine 320:265-271. Omega-3 PUFAs have also been shown to inhibit inflammation by inhibition of autoamplification of neutrophil inflammatory response by decreasing the formation of the dihydroxy fatty acid leukotriene B₄. Sperling (1993) J. Clin. Invest. 91:651-660.

4. Anti-Cancer Benefits of Omega-3 Polyunsaturated Fatty Acids

An important factor providing evidence that dietary fats can have a significant effect on tumorigenesis is data which suggest that the type of fat in the diet may be as important as the quantity of fat in mediating tumor promotion. In this regard, a great deal of attention has been focused on PUFAs. Although the precise mechanisms responsible for the effects of PUFAs are unknown, it has been suggested that PUFA effects are mediated through arachidonic acid, possibly via prostaglandins, HETEs and leukotrienes.

It has long been known that dietary omega-3 PUFAs are very effective in depressing tissue arachidonic acid content, and that the long chain omega-3 PUFAs are more effective than α-linolenic acid. Whelan, J., Broughton, K. S. and Kinsella, J. E., Lipids, Vol. 26, 119-126 (1991); Hwang, D. H., Boudreau, M. and Chanmugan, P., J. Nutr., Vol. 118, 427-437 (1988).

In addition, diets containing omega-3 PUFAs, particularly those found in fish oils (i.e., eicosapentaenoic acid (EPA) and docosahexaenoic acid (DHA)), are reported to diminish tumor formation and promotion, and omega-3 PUFA intake is negatively correlated with chemically-induced tumorigenesis. Braden, L. M. and Carroll, K. K., Lipids 21:285-288, 1986; Reddy, B., and Maruyama, H., Cancer Res. 46:3367-3370, 1986.

Tissue arachidonic acid content is correlated with eicosanoid biosynthesis. Li, B. Y., Birdwell, C. and Whelan, J., J. Lipid. Res., Vol. 35, 1869-1877 (1994).

Eicosapentaenoic acid levels in colonic mucosal phospholipids are negatively associated with indices of cell proliferation. Lee, D. -Y. K., Lupton, J. R., Aukema, H. M. and Chapkin, R. S., J. Nutr., Vol. 123, 1808-1917 (1993).

Conversely, arachidonic acid content in colonic mucosal phospholipids is associated with higher indices of cell proliferation. Lee, D. -Y. K., Lupton, J. R., Aukema, H. M. and Chapkin, R. S., J. Nutr., Vol. 123, 1808-1917 (1993).

More recently, Paulson et al. showed that a fish oil derived concentrate of eicosapentaenoic acid (EPA) and docosahexaenoic acid (DHA) decreased intestinal polyp formation and growth in Δ716 Apc knockout Min/+ mice. Carcinogenesis, Vol. 18, 1905-1910 (1997). Similarly, Oshima et al. showed that dietary DHA-ethyl ester reduced intestinal polyp development in 716 Apc knockout Min/+ mice. Carcinogenesis, Vol. 16, 2605-2607 (1995). Moser, A. R., Luongo, C., Gould, K. A., McNeley, M. K., Shoemaker, A. R., Dove, W. F., Eur. J. Cancer, 31A(7-8), 1061-1064 (1995).

European patent application No. 0 440 307 A2 discloses compositions for use in the treatment of breast cancer. The disclosed compositions contain one or more metabolites of α-linolenic acid and one or more metabolites of linoleic acid.

International Application No. 97/39749 describes methods for the prevention and treatment of cachexia and anorexia. Cachexia and anorexia are said to be common conditions among cancer patients whose diseases have progressed to metastatic cancer. The disclosed methods involve administering to an individual an oil blend containing n-6 and omega-3 fatty acids, a source of amino-nitrogen which includes branched-chain amino acids, and an antioxidant component.

International Publication No. WO/01/17517 discloses metabolite(s) of α-linolenic acid, such as stearidonic acid (18:4 n-3), eicosatetraenoic acid (20:4 n-3), docosapentaenoic acid (22:5 n-3) and mixtures thereof, especially metabolites including stearidonic acid for treatment and prevention of cancer.

U.S. Pat. No. 5,886,037 discloses food compositions for treatment of various diseases which may be associated with the metabolic syndrome (syndrome X), including hyperlipoproteinaemia, obesity, hyperuricemia, hypertension, fatty liver, diabetes type II, insulin resistance and atherosclerotic vascular disease. The disclosed compositions contain medium-chain fatty acids and omega-3 polyunsaturated long chain fatty acids.

U.S. Pat. No. 5,158,975 describes the use of stearidonic acid for prevention and treatment of inflammatory conditions, including allergic disorders, skin disorders, rheumatic disorders, and those following trauma, shock and pathologies. Stearidonic acid (SDA) and its metabolites, EPA and DHA, are said to inhibit biosynthesis of leukotrienes which are involved in the inflammation process.

### BRIEF DESCRIPTION OF THE INVENTION

1. General

The present invention relates to combinations of omega-3 polyunsaturated fatty acids (PUFAs) and cyclooxygenase inhibitors, and in particular combinations of omega-3 polyunsaturated fatty acids selective cyclooxygenase-2 (COX-2) inhibitors. In combination, omega-3 PUFAs and cyclooxygenase inhibitors are administered to subjects in need of treatment or prevention of cardiovascular disease, inflammation, diabetes or cancer, for treatment or prevention of cardiovascular disease, inflammation, diabetes or cancer.

An omega-3 PUFA taken in combination with a cyclooxygenase-2 (COX-2) selective inhibiting drug confers a cardiovascular-protective benefit directly to users of those COX-2 inhibiting drugs, especially elderly subjects. In the elderly, decreased baroreceptor responsiveness, coupled with decreased arterial compliance, accounts for frequent orthostatic hypotension. NSAIDs and cyclooxygenase-2 selective inhibitors may cause renal sodium (Na⁺) retention and inhibit prostaglandin-induced vasodilation. However, a cardiovascular-protective benefit would also be conferred to younger subjects and would be sustained through those subjects' later stages of life.

Various types of evidence including results from laboratory-based investigations, observational studies, and clinical trials suggest that consumption of omega-3 polyunsaturated fatty acids (omega-3 PUFA), from either commercially available fish oil supplements or certain high-fat fish, may reduce blood pressure. The mechanisms by which omega-3 PUFA s may reduce blood pressure remain uncertain. It is known that certain omega-3 PUFAs, particularly DHA and EPA, inhibit the desaturation of n-6 fatty acids, and therefore inhibit arachidonic acid production. While not wishing to be bound by theory, it is postulated that omega-3 PUFAs may decrease vascular resistance by decreasing the production of the vasoconstrictor thromboxane A₂ (TXA₂) and thereby creating a decrease in the ratio of TXA₂ and the vasodilator prostacyclin, PGI₂. It has been suggested that some COX-2 selective inhibitors may reduce in vivo production of both thromboxane A₂ (TXA₂), a pro-inflammatory prostaglandin, and prostacyclin, PGI₂, an anti-inflammatory prostaglandin. While not wishing to be bound by any theory, it is believed that the combination of omega-3 PUFAs and COX-2 selective inhibitors may tend to maintain a hoeostatic ratio of thromboxane A₂ (TXA₂) and prostacyclin, PGI₂ in a subject, thereby conferring a cardiovascular benefit over administration of a COX-2 selective inhibitor alone.

A combination of omega-3 PUFAs and COX-2 selective inhibitors is believed to impart additive anti-inflammatory benefits over administration of either component individually. While COX-2 selective inhibitors exert anti-inflammatory effects by preventing COX-2 activity and thereby decreasing prostaglandin production, omega-3 PUFAs inhibit inflammation through suppression of monocyte production of the polypeptide cytokines interleukin-1 (IL-1β and IL-1α) and tumor necrosis factor. Because different mediators of inflammation are suppressed by omega-3 PUFAs and COX-2 selective inhibitors, it is believed that an increased anti-inflammatory response will be realized through the administration of a combination of both omega-3 PUFAs and COX-2 selective inhibitors.

Many studies have shown that dietary fish oil rich in omega-3 PUFAs, particularly eicosapentaenoic acid (EPA) and docosahexaenoic acid (DHA), has a hypotriglyceridemic effect but the mechanism of its action is not fully understood. In addition, elevated levels of IL-1 over basal levels have been implicated in mediating or exacerbating a number of disease states including type I and type II diabetes. TNF-α and IL-1 appear to play a role in pancreatic B cell destruction and diabetes. Pancreatic beta. cells produce insulin which helps mediate blood glucose homeostasis. Deterioration of pancreatic beta. cells often accompanies type I diabetes. Pancreatic beta cell functional abnormalities may occur in patients with type II diabetes. Since omega-3 PUFAs suppress TNF-α and IL-1 production by monocytes, it is believed that omega-3 PUFAs would be beneficial in the treatment or prevention of diabetes.

Further, omega-3 PUFAs, like COX-2 inhibiting drugs, have been shown to possess anti-cancer properties in animal models. It is thus believed that an omega-3 PUFA and COX-2 inhibiting drug combination product would provide greater efficacy when compared to the COX-2 inhibiting drug alone.

2. Vertebrate Fatty Acid Metabolism

There are two types of essential fatty acids (EFAs), the n-3 (or ω-3) type derived from α-linolenic acid and the n-6 (or ω-6) type derived from linoleic acid. The starting polyunsaturated fatty acids (PUFAs) of these metabolic pathways (i.e., α-linolenic acid and linoleic acid) cannot be produced in vertibrates, and therefore must be obtained in the diet.

### DESCRIPTION OF THE PREFERRED EMBODIMENTS

1. Definitions

As used herein, n-3 PUFA means an omega-3 polyunsaturated fatty acid, either naturally occurring, produced recombinantly or produced synthetically. Various naming conventions exist for describing unsaturated fatty acids. Trivial names (e.g. α-linolenic acid) are sometimes used to describe polyunsaturated fatty acids. Abbreviations (e.g. ALA for α-linolenic acid) are also often employed to identify a polyunsaturated fatty acid. A somewhat obsolete convention uses the Greek symbol Δ (delta) to describe the position of the carbon in relation to the first double bond from the carboxylic moiety of the fatty acid (e.g. 18:3 Δ^{9,12,15}). The IUPAC nomenclature system also numbers the carbons from the carboxyl end of a fatty acid (e.g. cis 9,12,15-octadecatrienoic acid). Finally, the more popular modern convention uses the number of carbons in the chain, followed by a colon, and then the Greek symbol ω (omega) to describe the first double bond from the methyl end. Thus, ALA (cis 9,12,15-octadecatrienoic acid) is named. 18:3 ω-3 (eighteen carbons, three double bonds, the first double bond occurring at the third carbon from the methyl end). A name associated with a fatty acid herein is not intended to place a limitation thereon, and any other way of describing a polyunsaturated fatty acid molecule other than the name used herein is intended to fall within the scope of the present specification and claims. n-3 PUFAs include salts, esters and glyceride conjugates of omega-3 fatty acids.

The phrase “substantially all cis”, as applied to n-3 PUFAs means preferably in at least 90% of the compound, the hydrogen atoms are on the same side of the double bond of the polyunsaturated fatty acid, and more preferably in at least 95% of the compound, the hydrogen atoms are on the same side of the double bond of the polyunsaturated acid, and most preferably in at least 99% of the compound, the hydrogen atoms are on the same side of the double bond of the polyunsaturated acid.

The term “cyclooxygenase-2 inhibitor” denotes a compound preferably inhibiting cyclooxygenase-2 relative to inhibition of cyclooxygenase-1. Preferably, it includes compounds which have a cyclooxygenase-2 IC₅₀ of less than about 0.2 μM, and also have a selectivity ratio of cyclooxygenase-2 inhibition over cyclooxygenase-1 inhibition of at least 50, and more preferably of at least 100. Even more preferably, the compounds have a cyclooxygenase-1 IC₅₀ of greater than about 1 μM, and more preferably of greater than 10 μM.

The term “purified” means partially purified and/or completely purified. Thus a “purified composition” may be either partially purified or completely purified. An extract of a naturally occurring cyclooxygenase-2 inhibitor may be partially purified or purified or synthesized.

A pharmaceutical combination of the present invention is directed to a combination suitable for the treatment of hypertrophy, hypertension, congestive heart failure, ischemic heart disease, ischemia reperfusion injury and cellular dysfunction. The pharmaceutical combination comprises a pharmaceutically acceptable carrier and a combination selected from n-3 PUFAs and COX-2 selective inhibitors. A pharmaceutically acceptable carrier includes, but is not limited to, physiological saline, Ringer's, phosphatesolution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfiric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the combination of the invention are the isomeric forms and tautomers of the described compounds and the pharmaceutically-acceptable salts thereof. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

Effective amount means the dose or effective amount to be administered to a patient and the frequency of administration to the subject which is readily determined by one or ordinary skill in the art, by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

Co-administration and co-administered mean both taken in a single delivery vehicle, taken together contemporaneously, or taken within a period of time sufficient to receive a beneficial effect from both of the constituent agents of the combination.

The phrase “therapeutically-effective” is intended to qualify the amount of each agent which will achieve the goal of improvement in disease severity and the frequency of incidence over treatment of each agent by itself, while avoiding adverse side effects typically associated with alternative therapies.

The terms “treating” or “to treat” means to alleviate symptoms, eliminate the causation either on a temporary or permanent basis, or to prevent or slow the appearance of symptoms. The term “treatment” includes alleviation, elimination of causation of or prevention of hypertrophy, hypertension, congestive heart failure, ischemic heart disease, ischemia reperfusion injury and cellular dysfunction partial or total inhibition of growth, spreading or metastasis of benign tumors, cancerous tumors and polyps, as well as partial or total destruction of tumor and polyp cells. The term “prevention” includes either preventing the onset of clinically evident of hypertrophy, hypertension, congestive heart failure, ischemic heart disease, ischemia reperfusion injury and cellular dysfunction, tumors or polyps altogether or preventing the onset of a preclinically evident stage of tumor or polyp development in individuals at risk. The term “prevention” also includes prevention of initiation for malignant cells or to arrest or reverse the progression of premalignant cells to malignant cells. This includes those at risk for developing tumors and/or polyps.

The term “subject” for purposes of treatment includes any human or animal subject who has any one of the known CVD, neoplasia, tumor disorders, or, inflammation related disorders, and is preferably a human subject. For methods of prevention, the subject is any human or animal subject, and preferably is a human subject who is at risk for obtaining CVD, an intestinal cancer or neoplasia-related disorder, either benign or malignant, including metastasis, or an inflammation related disorder. The subject may be at risk due to genetic predisposition, sedentary lifestyle, diet, exposure to carcinogenic agents, exposure to pathogenic agents and the like. Non-exclusive cancers and neoplastic conditions include: malignant tumor growth selected from the group consisting of acral lentiginous melanoma, actinic keratoses, acute lymphocytic leukemia, acute myeloid leukemia, adenocarcinoma, adenoid cycstic carcinoma, adenomas, adenosarcoma, adenosquamous carcinoma, anal canal cancer, anal cancer, anorectum cancer, astrocytic tumors, bartholin gland carcinoma, basal cell carcinoma, biliary cancer, bone cancer, bone marrow cancer, brain cancer, breast cancer, bronchial cancer, bronchial gland carcinomas, carcinoids, carcinoma, carcinosarcoma, cholangiocarcinoma, chondosarcoma, choriod plexus papilloma/carcinoma, chronic lymphocytic leukemia, chronic myeloid leukemia, clear cell carcinoma, colon cancer, colorectal cancer, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, esophageal cancer, Ewing's sarcoma, eye and orbit cancer, female genital cancer, focal nodular hyperplasia, gallbladder cancer, gastric antrum cancer, gastric fundus cancer, gastrinoma, germ cell tumors, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, kidney and renal pelvic cancer, large cell carcinoma, large intestine cancer, larynx cancer, leiomyosarcoma, lentigo maligna melanomas, leukemia, liver cancer, lung cancer, lymphoma, male genital cancer, malignant melanoma, malignant mesothelial tumors, medulloblastoma, medulloepithelioma, melanoma, meningeal cancer, mesothelial cancer, metastatic carcinoma, mouth cancer, mucoepidermoid carcinoma, multiple myeloma, muscle cancer, nasal tract cancer, nervous system cancer, neuroblastoma, neuroepithelial adenocarcinoma nodular melanoma, non-epithelial skin cancer, non-Hodgkin's lymphoma, oat cell carcinoma, oligodendroglial cancer, oral cavity cancer, osteosarcoma, ovarian cancer, pancreatic cancer, papillary serous adenocarcinoma, penile cancer, pharynx cancer, pituitary tumors, plasmacytoma, prostate cancer, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, stomach cancer, striated muscle cancer, submesothelial cancer, superficial spreading melanoma, T cell leukemia, testicular cancer, thyroid cancer, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, and Wilms tumor.

The term neoplasia includes both benign and cancerous tumors and growths.

The pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition should be at or near body temperature.

In a preferred embodiment of the present invention, the ω-3 fatty acids are selected from the group of metabolic n-3 PUFAs. Omega 3 fatty acids are a subset of methylene-interrupted polyenes. Polyenoic fatty acids (also called polyunsaturated fatty acids, PUFA) usually having 2 or more cis double bonds which are most frequently separated from each other by a single methylene group. The omega 3 fatty acids have a common structural feature: CH₃(CH₂)CH═R, where R is a carbon chain from 15 to 19 carbons in length, terminating in a carboxylic acid group. In nature, n-3 PUFAs are typically all cis, that is to say all of the hydrogens occur at the same side of the carbon-carbon double bonds. There are examples, such as bovine milk, that contain a small fraction of trans isomers of n-3 PUFAs. In addition, synthetic formulations of n-3 PUFAs may be mixtures of cis and trans isomers. It is preferred in the present invention that the n-3 PUFAs used are substantially all cis.

A simplified desaturation and elongation pathway for n-3 PUFAs in mammals are shown below, in Table 1.

TABLE 1
Mammalian Metabolic Pathway for α-linolenic acid.
18:3 n-3 (α.:-linolenic acid or ALA) CH₃(CH₂CH═CH)₃(CH₂)₇COOH
9,12,15-octadecatrienoic acid
δ-6 desaturase
↓
18:4 n-3 (Stearidonic acid or SDA) CH₃CH₂(CH═CHCH₂)₄(CH₂)₃COOH
6,9,12,15-octadecatetranoic acid
chain elobgation
↓
20:4 n-3 (Eicosatetraenoic acid) CH₃CH₂(CH═CHCH₂)₄(CH₂)₅COOH
8,11,14,17-eicosatetraenoic acid
δ-5 desaturase
↓
20:5 n-3 (Eicosapentaenoic acid or EPA)
CH₃(CH₂CH═CH)₅(CH₂)₃COOH
cis 5,8,11,14,17-eicosapentaenoic acid
chain elongation
↓
chain elongation
↓
22:5 n-3 (Docosapentaenoic acid) CH₃CH₂(CH═CHCH₂)₅(CH₂)₄COOH
cis 7,10,13,16,19 docosapentaenoic acid
chain elongation
↓
chain elongation
↓
24:5 n-3
δ-6 desaturase
↓
24:6 n-3
δ-oxidation

While it has become more common to use the omega naming convention rather than the IUPAC name for describing an unsaturated fatty acid, the names of the desaturase enzymes responsible for removing hydrogen (and thus adding double bonds) are named using the delta naming convention (e.g. Δ-6 desaturase adds a double bond at the Δ⁶ position).

The preferred n-3 PUFAs of the present invention are selected from the group of n-3 PUFAs from the mammalian metabolic pathway of Table 1.

Stearidonic acid (SDA; 18:4 ω-3) is a preferred n-3 PUFA of the present invention. SDA is the precursor of the long-chain n-3 PUFAs, EPA (20:5 n-3) and DHA (22:6 n-3). SDA is the product of delta-6 desaturation of α-linolenic acid (ALA; 18:3 n-3). The delta-6 desaturase is considered to be the rate-limiting step in the n-3 PUFA synthetic pathway. It has been discovered that SDA is on par with or even superior to EPA and/or DHA as an anti-inflammatory and anti-cancer agent. ALA, on the other hand, appears to be less efficacious. SDA can be categorized as a “pro”-EPA/DHA fatty acid.

One source of n-3 PUFAs are micro-algal-derived oils enriched in long-chain n-3 PUFAs. For example the commercial product NEUROMINS available from Martek Biosciences Corporation, 6480 Dobbin Road Columbia Md. 21045 contains about 50% DHA

Fish liver oils, especially those fish derived oils from cold water, deep swimming “fatty” species are a known source of n-3 PUFAs, especially EPA and DHA. EPA- or DHA-ethyl ester (EE) can purified from fish oil to a high degree of purity (90-95%). SDA-EE, EPA-EE or DHA-EE, either alone or in combination are also contemplated within the scope of the present invention. Also, triglycerides enriched in SDA, EPA and DHA may be used within the intended scope of the present invention. Triglycerides containing ALA, SDA, n-3 DPA, EPA and DHA are saponified, and the released EPA and DHA are concentrated and then re-esterified to triglyceride.

Table 2 shows the compound designations A1 through A-6 for the compounds of the present invention, the chemical names, trivial names (where appropriate), and formulas. Table 2 represents the preferred n-3 PUFAs of the present invention.

TABLE 2
Examples of n-3 PEFAs as Embodiments

| Compound | |
|---|---|
| Number | Name, Trivial Name, and Formula |
| A-1 | all cis 9,12,15-octadecatrienoic acid (α.-linoleic acid or |
| | ALA) 18:3 ω3 |
| | CH₃(CH₂CH═CH)₃(CH₂)₂COOH |
| A-2 | all cis 6,9,12,15-octadecatetraenoic acid (Stearidonic acid |
| | or SDA) 18:4 ω-3 |
| | CH₃CH₂(CH═CHCH₂)₄(CH₂)₃COOH |
| A-3 | all cis 8,11,14,17-eicosatetraenoic acid (Eicosatetraenoic |
| | acid) 20:4 ω-3 |
| | CH₃CH₂(CH═CHCH₂)₄(CH₂)₅COOH |
| A-4 | all cis (5,8,11,14,17-eicosapentaenoic acid (EPA) 20:5 ω-3 |
| | CH₃(CH₂CH═CH)₅(CH₂)₃COOH |
| A-5 | all cis 7,10,13,16,19 docosapentaenoic acid (Docosapenta- |
| | enoic acid) 22:5 ω-3 |
| | CH₃CH₂(CH═CHCH₂)₅(CH₂)₄COOH |
| A-6 | all cis 4,7,10,13,16,19-docosahexanoic acid (DHA) |
| | 22:6 ω-3 |
| | CH₃(CH₂CH═CH)₆CH₂CH₂COOH |

The physical properties of fatty acids are determined by chain length, degree of unsaturation, and chain branching. Table 2 comprises the preferred n-3 PUFAs of the present invention, and are referred to herein collectively as Group I. Other compounds of Group I include, for example, SDA ethyl ester, EPA ethyl ester, DHA ethyl ester, and triglycerides enriched, for example, in SDA, EPA or DHA.

In an embodiment of the present invention, a cyclooxygenase-2 selective inhibitor is combined with the n-3 PUFA compounds of the present invention.

In another embodiment of the invention the cyclooxygenase-2 selective inhibitor can be, for example, the COX-2 selective inhibitor meloxicam, Formula B-1 (CAS registry number 71125-38-7) or a pharmaceutically acceptable salt or prodrug thereof.

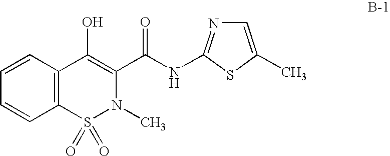

In yet another embodiment of the invention the cyclooxygenase-2 selective inhibitor is the COX-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3) or a pharmaceutically acceptable salt or prodrug thereof.

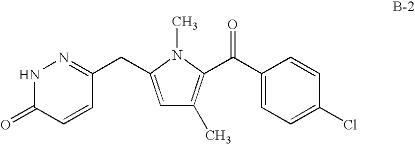

In a preferred embodiment of the invention the cyclooxygenase-2 selective inhibitor is preferably of the chromene structural class that is a substituted benzopyran or a substituted benzopyran analog, and even more preferably selected from the group consisting of substituted benzothiopyrans, dihydroquinolines, or dihydronaphthalenes having the general Formula II shown below and possessing, by way of example and not limitation, the structures disclosed in Table 3, including the diastereomers, enantiomers, racemates, tautomers, salts, esters, amides and prodrugs thereof. Furthermore, benzopyran COX-2 selective inhibitors useful in the practice of the present invention are described in U.S. Pat. Nos. 6,034,256 and 6,077,850 herein incorporated by reference.

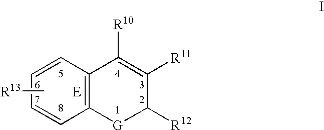

wherein G is selected from the group consisting of O or S or NR^{a}; wherein R^{a} is alkyl;

wherein R¹⁰ is selected from the group consisting of H and aryl

wherein R¹¹ is selected from the group consisting of carboxyl, aminocarbonyl, alkylsulfonylaminocarbonyl and alkoxycarbonyl;

wherein R¹² is selected from the group consisting of haloalkyl, alkyl, aralkyl, cycloalkyl and aryl optionally substituted with one or more radicals selected from alkylthio, nitro and alkylsulfonyl; or

wherein R¹³ is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R¹³ together with ring E forms a naphthyl radical; or an isomer or pharmaceutically acceptable salt thereof.

Formula II is:

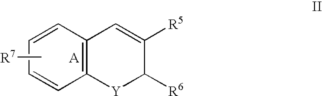

wherein:

Y is selected from the group consisting of O or S or NR^{b};

R^{b} is alkyl;

R⁵ is selected from the group consisting of carboxyl, aminocarbonyl, alkylsulfonylaminocarbonyl and alkoxycarbonyl;

R⁶is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

R⁷is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁷ together with ring A forms a naphthyl radical;

or an isomer or pharmaceutically acceptable salt thereof.

The cyclooxygenase-2 selective inhibitor may also be a compound of Formula II, wherein:

Y is selected from the group consisting of oxygen and sulfur;

R⁵ is selected from the group consisting of carboxyl, lower alkyl, lower aralkyl and lower alkoxycarbonyl;

R⁶ is selected from the group consisting of lower haloalkyl, lower cycloalkyl and phenyl; and

R⁷ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or

wherein R⁷ together with ring A forms a naphthyl radical;

or an isomer or pharmaceutically acceptable salt thereof.

The cyclooxygenase-2 selective inhibitor may also be a compound of Formula II, wherein:

R⁵is carboxyl;

R⁶is lower haloalkyl; and

R⁷is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R⁷ together with ring A forms a naphthyl radical;

or an isomer or pharmaceutically acceptable salt thereof.

The cyclooxygenase-2 selective inhibitor may also be a compound of Formula II, wherein:

R⁶is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R⁷is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R² together with ring A forms a naphthyl radical;

or an isomer or pharmaceutically acceptable salt thereof.

The cyclooxygenase-2 selective inhibitor may also be a compound of Formula II, wherein:

R⁶is selected from the group consisting trifluoromethyl and pentafluoroethyl; and

R⁷ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R⁷ together with ring A forms a naphthyl radical;

or an isomer or prodrug thereof.

The cyclooxygenase-2 selective inhibitor of the present invention can also be a compound having the structure of Formula III:

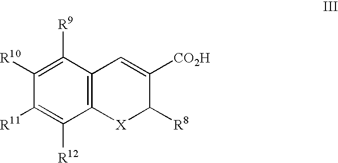

wherein:

X is selected from the group consisting of O and S;

R⁸ is lower haloalkyl;

R⁹ is selected from the group consisting of hydrido, and halo;

R¹⁰ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6- membered nitrogen-containing heterocyclosulfonyl;

R¹¹ is selected from the group consisting of hydrido, lower alkyl, halo, lower alkoxy, and aryl; and

R¹² is selected from the group consisting of the group consisting of hydrido, halo, lower alkyl, lower alkoxy, and aryl;

or an isomer or prodrug thereof.

The cyclooxygenase-2 selective inhibitor can also be a compound of having the structure of Formula III, wherein

R⁸ is selected from the group consisting of trifluoromethyl and pentafluoroethyl;

R⁹ selected from the group consisting of hydrido, chloro, and fluoro;

R¹⁰ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methyl sulfonyl, and morpholinosulfonyl;

R¹¹ is selected from the group consisting of hydrido, methyl, ethyl, isopropyl, tert-butyl, chloro, methoxy, diethyl amino, and phenyl; and

R¹² is selected from the group consisting of hydrido, chloro, bromo, fluoro, methyl, ethyl, tert-butyl, methoxy, and phenyl;

or an isomer or prodrug thereof.

TABLE 3
Examples of Chromene Cox-2 Selective Inhibitors as Embodiments

| Compound | |
|---|---|
| Number | Structural Formula |
| B-3 | 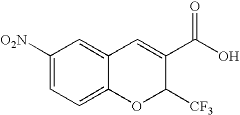 |
| | 6-Nitro-2-trifluoromethyl-2H-1- |
| | benzopyran-3-carboxylic acid |
| B-4 | 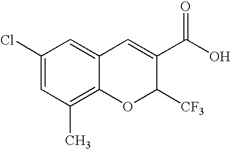 |
| | 6-Chloro-8-methyl-2-trifluoromethyl- |
| | 2H-1-benzopyran-3-carboxylic acid |
| B-5 | 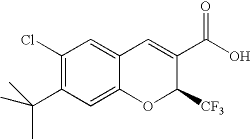 |
| | ((S)-6-Chloro-7-(1,1-dimethylethyl)-2-(trifluoromethyl- |
| | 2H-1-benzopyran-3-carboxylic acid |
| B-6 | 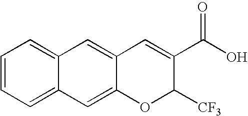 |
| | 2-Trifluoromethyl-2H-naphtho[2,3-b] |
| | pyran-3-carboxylic acid |
| B-7 | 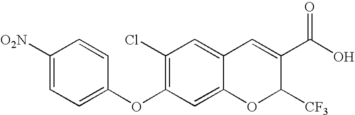 |
| | 6-Chloro-7-(4-nitrophenoxy)-2-(trifluoromethyl)-2H-1- |
| | benzopyran-3-carboxylic acid |
| B-8 | 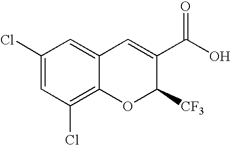 |
| | ((S)-6,8-Dichloro-2-(trifluoromethyl)- |
| | 2H-1-benzopyran-3-carboxylic acid |
| B-9 | 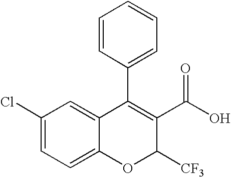 |
| | 6-Chloro-2-(trifluoromethyl)-4-phenyl-2H- |
| | 1-benzopyran-3-carboxylic acid |
| B-10 | 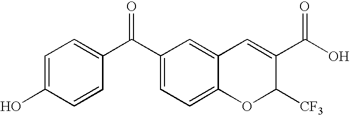 |
| | 6-(4-Hydroxybenzoyl)-2-(trifluoromethyl)- |
| | 2H-1-benzopyran-3-carboxylic acid |
| B-11 | 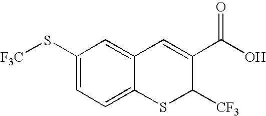 |
| | 2-(Trifluoromethyl)-6-[(trifluoromethyl)thio]- |
| | 2H-1-benzothiopyran-3-carboxylic acid |
| B-12 | 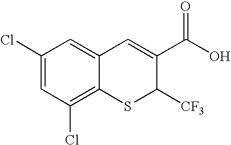 |
| | 6,8-Dichloro-2-trifluoromethyl-2H-1- |
| | benzothiopyran-3-carboxylic acid |
| B-13 | 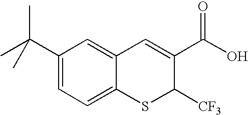 |
| | 6-(1,1-Dimethylethyl)-2-(trifluoromethyl)- |
| | 2H-1-benzothiopyran-3-carboxylic acid |
| B-14 | 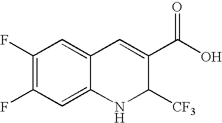 |
| | 6,7-Difluoro-1,2-dihydro-2-(trifluoromethyl)- |
| | 3-quinolinecarboxylic acid |
| B-15 | 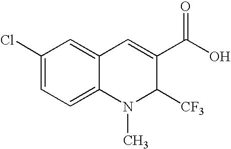 |
| | 6-Chloro-1,2-dihydro-1-methyl-2-(trifluoromethyl)- |
| | 3-quinolinecarboxylic acid |
| B-16 | 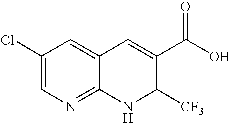 |
| | 6-Chloro-2-(trifluoromethyl)-1,2-dihydro |
| | [1,8]naphthyridine-3-carboxylic acid |
| B-17 | 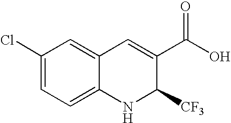 |
| | ((S)-6-Chloro-1,2-dihydro-2-(trifluoromethyl)- |
| | 3-quinolinecarboxylic acid |

The compound shown as the structure in FIG. B-8, above, is a preferred chromene-type cyclooxygenase-2 selective inhibitor. The sodium salt form of the compound is preferred. Further information about compound shown as the structure in FIG. B-8 can be found in U.S. Pat. No. 6,034,256.

Specific compounds that are useful for the cyclooxygenase-2 selective inhibitor include:

a1) 8-acetyl-3-(4-fluorophenyl)-2-(4-methylsulfonyl)phenyl-imidazo(1,2-a)pyridine;

a2) 5,5-dimethyl-4-(4-methylsulfonyl)phenyl-3-phenyl-2-(5H)-furanone;

a3) 5-(4-fluorophenyl)-1-[4-(methylsulfonyl)phenyl]-3-(trifluoromethyl)pyrazole;

a4) 4-(4-fluorophenyl)-5-[4-(methylsulfonyl)phenyl]-1-phenyl-3-(trifluoromethyl)pyrazole;

a5) 4-(5-(4-chlorophenyl)-3-(4-methoxyphenyl)-1H-pyrazol-1-yl)benzenesulfonamide

a6) 4-(3,5-bis(4-methylphenyl)-1H-pyrazol-1-yl)benzenesulfonamide;

a7) 4-(5-(4-chlorophenyl)-3-phenyl-1H-pyrazol-1-yl)benzenesulfonamide;

a8) 4-(3,5 -bis(4-methoxyphenyl)-1H-pyrazol- 1-yl)benzenesulfonamide;

a9) 4-(5-(4-chlorophenyl)-3-(4-methylphenyl)-1H-pyrazol-1-yl)benzenesulfonamide;

a10) 4-(5-(4-chlorophenyl)-3-(4-nitrophenyl)-1H-pyrazol-1-yl)benzenesulfonamide;

b1) 4-(5-(4-chlorophenyl)-3-(5-chloro-2-thienyl)-1H-pyrazol-1-yl)benzenesulfonamide;

b2) 4-(4-chloro-3,5-diphenyl-1H-pyrazol-1-yl)benzenesulfonamide

b3) 4-[5-(4-chlorophenyl)-3-(trifluoromethyl)-1H-pyrazol-1-yl]benzenesulfonamide;

b4) 4-[5-pbenyl-3-(trifluoromethyl)-1H-pyrazol-1-yl]benzenesulfonamide;

b5) 4-[5-(4-fluorophenyl)-3-(trifluoromethyl)-1H-pyrazol-1-yl]benzenesulfonamide;

b6) 4-[5-(4-methoxyphenyl)-3-(trifluoromethyl)-1H-pyrazol-1-yl]benzenesulfonamide;

b7) 4-[5-(4-chlorophenyl)-3-(difluoromethyl)-1H-pyrazol-1-yl]benzenesulfonamide;

b8) 4-[5-(4-methylphenyl)-3-(trifluoromethyl)-1H-pyrazol-1-yl]benzenesulfonamide;

b9) 4-[4-chloro-5-(4-chlorophenyl)-3-(trifluoromethyl)-1H-pyrazol-1-yl]benzenesulfonamide;

b10) 4-[3-(difluoromethyl)-5-(4-methylphenyl)-1H-pyrazol-1-yl]benzenesulfonamide;

c1) 4-[3-(difluoromethyl)-5-phenyl-1H-pyrazol-1-yl]benzenesulfonamide;

c2) 4-[3-(difluoromethyl)-5-(4-methoxyphenyl)-1H-pyrazol-1-yl]benzenesulfonamide;

c3) 4-[3-cyano-5-(4-fluorophenyl)-1H-pyrazol-1-yl]benzenesulfonamide;

c4) 4-[3 -(difluoromethyl)-5-(3-fluoro-4-methoxyphenyl)-1H-pyrazol-1-yl]benzenesulfonamide;

c5) 4-[5-(3-fluoro-4-methoxyphenyl)-3-(trifluoromethyl)-1H-pyrazol-1-yl]benzenesulfonamide;

c6) 4-[4-chloro-5-phenyl-1H-pyrazol-1-yl]benzenesulfonamide;

c7) 4-[5-(4-chlorophenyl)-3-(hydroxymethyl)-1H-pyrazol-1-yl]benzenesulfonamide;

c8) 4-[5-(4-(N,N-dimethylamino)phenyl)-3-(trifluoromethyl)-1H-pyrazol-1-yl]benzenesulfonamide;

c9) 5-(4-fluorophenyl)-6-[4-(methylsulfonyl)phenyl]spiro[2.4]hept-5-ene;

c10) 4-[6-(4-fluorophenyl)spiro[2.4]hept-5-en-5-yl]benzenesulfonamide;

d1) 6-(4-fluorophenyl)-7-[4-(methylsulfonyl)phenyl]spiro[3.4]oct-6-ene;

d2) 5-(3-chloro-4-methoxyphenyl)-6-[4-(methylsulfonyl)phenyl]spiro [2.4]hept-5-ene;

d3) 4-[6-(3-chloro-4-methoxyphenyl)spiro[2.4]hept-5-en-5-yl]benzenesulfonamide;

d4) 5-(3,5-dichloro-4-methoxyphenyl)-6-[4-(methylsulfonyl)phenyl]spiro[2.4]hept-5-ene;

d5) 5-(3-chloro-4-fluorophenyl)-6-[4-(methylsulfonyl)phenyl]spiro[2.4]hept-5-ene;

d6) 4-[6-(3,4-dichlorophenyl)spiro[2.4]hept-5-en-5-yl]benzenesulfonamide;

d7) 2-(3-chloro-4-fluorophenyl)-4-(4-fluorophenyl)-5-(4-methylsulfonylphenyl)thiazole;

d8) 2-(2-chlorophenyl)-4-(4-fluorophenyl)-5-(4-methylsulfonylphenyl)thiazole;

d9) 5-(4-fluorophenyl)-4-(4-methylsulfonylphenyl)-2-methylthiazole;

d10) 4-(4-fluorophenyl)-5-(4-methylsulfonylphenyl)-2-trifluoromethylthiazole;

e1) 4-(4-fluorophenyl)-5-(4-methylsulfonylphenyl)-2-(2-thienyl)thiazole;

e2) 4-(4-fluorophenyl)-5-(4-methylsulfonylphenyl)-2-benzylaminothiazole;

e3) 4-(4-fluorophenyl)-5-(4-methylsulfonylphenyl)-2-(1-propylamino)thiazole;

e4) 2-[(3,5-dichlorophenoxy)methyl)-4-(4-fluorophenyl)-5-[4-(methylsulfonyl)phenyl]thiazole;

e5) 5-(4-fluorophenyl)-4-(4-methylsulfonylphenyl)-2-trifluoromethylthiazole;

e6) 1-methylsulfonyl-4-[1,1-dimethyl-4-(4-fluorophenyl)cyclopenta-2,4-dien-3-2yl]benzene;

e7) 4-[4-(4-fluorophenyl)-1,1-dimethylcyclopenta-2,4-dien-3-yl]benzenesulfonamide;

e8) 5-(4-fluorophenyl)-6-[4-(methylsulfonyl)phenyl]spiro[2.4]hepta-4,6-diene;

e9) 4-[6-(4-fluorophenyl)spiro[2.4]hepta-4,6-dien-5-yl]benzenesulfonamide;

e10) 6-(4-fluorophenyl)-2-methoxy-5-[4-(methylsulfonyl)phenyl]-pyridine-3-carbonitrile;

f1) 2-bromo-6-(4-fluorophenyl)-5-[4-(methylsulfonyl)phenyl]-pyridine-3-carbonitrile;

f2) 6-(4-fluorophenyl)-5-[4-(methylsulfonyl)phenyl]-2-phenyl-pyridine-3-carbonitrile;

f3) 4-[2-(4-methylpyridin-2-yl)-4-(trifluoromethyl)-1H-imidazol-1-yl]benzenesulfonamide;

f4) 4-[2-(5-methylpyridin-3-yl)-4-(trifluoromethyl)-1H-imidazol-1-yl]benzenesulfonamide;

f5) 4-[2-(2-methylpyridin-3-yl)-4-(trifluoromethyl)-1H-imidazol-1-yl]benzenesulfonamide;

f6) 3-[1-[4-(methylsulfonyl)phenyl]-4-(trifluoromethyl)-1H-imidazol-2-yl]pyridine;

f7) 2-[1-[4-(methylsulfonyl)phenyl-4-(trifluoromethyl)-1H-imidazol-2-yl]pyridine;

f8) 2-methyl-4-[1-[4-(methylsulfonyl)phenyl-4-(trifluoromethyl)-1H-imidazol-2-yl]pyridine;

f9) 2-methyl-6-[1-[4-(methylsulfonyl)phenyl-4-(trifluoromethyl)-1H-imidazol-2-yl]pyridine;

f10) 4-[2-(6-methylpyridin-3-yl)-4-(trifluoromethyl)-1H-imidazol-1-yl]benzenesulfonamide;

g1) 2-(3,4-difluorophenyl)-1-[4-(methylsulfonyl)phenyl]-4-(trifluoromethyl)-1H-imidazole;

g2) 4-[2-(4-methylphenyl)-4-(trifluoromethyl)-1H-imidazol-1-yl]benzenesulfonamide;

g3) 2-(4-chlorophenyl)-1-[4-(methylsulfonyl)phenyl]-4-methyl-1H-imidazole;

g4) 2-(4-chlorophenyl)-1-[4-(methylsulfonyl)phenyl]-4-phenyl-1H-imidazole;

g5) 2-(4-chlorophenyl)-4-(4-fluorophenyl)-1-[4-(methylsulfonyl)phenyl]-1H-imidazole;

g6) 2-(3-fluoro-4-methoxyphenyl)-1-[4-(methylsulfonyl)phenyl-4-(trifluoromethyl)-1H-imidazole;

g7) 1 -[4-(methylsulfonyl)phenyl]-2-phenyl-4-trifluoromethyl-1H-imidazole;

g8) 2-(4-methylphenyl)-1-[4-(methylsulfonyl)phenyl]-4-trifluoromethyl-1H-imidazole;

g9) 4-[2-(3 -chloro-4-methylphenyl)-4-(trifluoromethyl)-1H-imidazol-1-yl]benzenesulfonamide;

g10) 2-(3-fluoro-5-methylphenyl)-1-[4-(methylsulfonyl)phenyl]-4-(trifluoromethyl)-1H-imidazole;

h1) 4-[2-(3-fluoro-5-methylphenyl)-4-(trifluoromethyl)-1H-imidazol-1-yl]benzenesulfonamide;

h2) 2-(3-methylphenyl)-1-[4-(methylsulfonyl)phenyl]-4-trifluoromethyl-1H-imidazole;

h3) 4-[2-(3 -methylphenyl)-4-trifluoromethyl-1H-imidazol-1-yl]benzenesulfonamide;

h4) 1 -[4-(methylsulfonyl)phenyl]-2-(3-chlorophenyl)-4-trifluoromethyl-1H-imidazole;

h5) 4-[2-(3-chlorophenyl)-4-trifluoromethyl-1H-imidazol-1-yl]benzenesulfonamide;

h6) 4-[2-phenyl-4-trifluoromethyl-1H-imidazol-1-yl]benzenesulfonamide;

h7) 4-[2-(4-methoxy-3-chlorophenyl)-4-trifluoromethyl-1H-imidazol-1-yl]benzenesulfonamide;

h8) 1-allyl-4-(4-fluorophenyl)-3-[4-(methylsulfonyl)phenyl]-5-(trifluoromethyl)-1H-pyrazole;

h10) 4-[1-ethyl-4-(4-fluorophenyl)-5-(trifluoromethyl)-1H-pyrazol-3-yl]benzenesulfonamide;

i1) N-phenyl-[4-(4-luorophenyl)-3-[4-(methylsulfonyl)phenyl]-5-(trifluoromethyl)-1H-pyrazol-1-yl]acetamide;

i2) ethyl [4-(4-fluorophenyl)-3-[4-(methylsulfonyl)phenyl]-5-(trifluoromethyl)-1H-pyrazol-1-yl]acetate;

i3) 4-(4-fluorophenyl)-3-[4-(methylsulfonyl)phenyl]-1-(2-phenylethyl)-1H-pyrazole;

i4) 4-(4-fluorophenyl)-3-[4-(methylsulfonyl)phenyl]-1-(2-phenylethyl)-5-(trifluoromethyl)pyrazole;

i5) 1-ethyl-4-(4-fluorophenyl)-3-[4-(methylsulfonyl)phenyl]-5-(trifluoromethyl)-1H-pyrazole;

i6) 5-(4-fluorophenyl)-4-(4-methylsulfonylphenyl)-2-trifluoromethyl-1H-imidazole;

i7) 4-[4-(methylsulfonyl)phenyl]-5-(2-thiophenyl)-2-(trifluoromethyl)-1H-imidazole;

i8) 5-(4-fluorophenyl)-2-methoxy-4-[4-(methylsulfonyl)phenyl]-6-(trifluoromethyl)pyridine;

i9) 2-ethoxy-5-(4-fluorophenyl)-4-[4-(methylsulfonyl)phenyl]-6-(trifluoromethyl)pyridine;

i10) 5-(4-fluorophenyl)-4-[4-(methylsulfonyl)phenyl]-2-(2-propynyloxy)-6-(trifluoromethyl)pyridine;

j1) 2-bromo-5-(4-fluorophenyl)-4-[4-(methylsulfonyl)phenyl]-6-(trifluoromethyl)pyridine;

j2) 4-[2-(3-chloro-4-methoxyphenyl)-4,5-difluorophenyl]benzenesulfonamide;

j3) 1-(4-fluorophenyl)-2-[4-(methylsulfonyl)phenyl]benzene;

j4) 5-difluoromethyl-4-(4-methylsulfonylphenyl)-3-phenylisoxazole;

j5) 4-[3-ethyl-5-phenylisoxazol-4-yl]benzenesulfonamide;

j6) 4-[5-difluoromethyl-3-phenylisoxazol-4-yl]benzenesulfonamide;

j7) 4-[5-hydroxymethyl-3-phenylisoxazol-4-yl]benzenesulfonamide;

j8) 4-[5-methyl-3-phenylisoxazol-4-yl]benzenesulfonamide;

j9) 1-[2-(4-fluorophenyl)cyclopenten-1-yl]-4-(methylsulfonyl)benzene;

j10) 1-[2-(4-fluoro-2-methylphenyl)cyclopenten-1-yl]-4-(methylsulfonyl)benzene;

k1) 1-[2-(4-chlorophenyl)cyclopenten-1-yl]-4-(methylsulfonyl)benzene;

k2) 1-[2-(2,4-dichorophenyl)cyclopenten-1-y]-4-(methylsulfonyl)benzene;

k3) 1-[2-(4-trifluoromethylphenyl)cyclopenten-1-yl]-4-(methylsulfonyl)benzene;

k4) 1-[2-(4-methylthiophenyl)cyclopenten-1-yl]-4-(methylsulfonyl)benzene;

k5) 1-[2-(4-fluorophenyl)-4,4-dimethylcyclopenten-1-yl]-4-(methylsulfonyl)benzene;

k6) 4-[2-(4-fluorophenyl)-4,4-dimethylyclopenten-1-yl]benzenesulfonamide;

k7) 1-[2-(4-chlorophenyl)-4,4-dimethylcyclopenten-1-yl]-4-(methylsulfonyl)benzene;

k8) 4-[2-(4-chlorophenyl)-4,4-dimethylcyclopenten-1-yl]benzenesulfonamide;

k9) 4-[2-(4-fluorophenyl)cyclopenten-1-yl]benzenesulfonamide;

k10) 4-[2-(4-chlorophenyl)cyclopenten-1-yl]benzenesulfonamide;

l1) 1-[2-(4-methoxyphenyl)cyclopenten-1-yl]-4-(methylsulfonyl)benzene;

l2) 1-[2-(2,3-difluorophenyl)cyclopenten-1-yl]-4-(methylsulfonyl)benzene;

l3) 4-[2-(3-fluoro-4-methoxyphenyl)cyclopenten-1-yl]benzenesulfonamide;

l4) 1-[2-(3-chloro-4-methoxyphenyl)cyclopenten-1-yl]-4-(methylsulfonyl)benzene;

l5) 4-[2-(3-chloro-4-fluorophenyl)cyclopenten-1-yl]benzenesulfonamide;

l6) 4-[2-(2-methylpyridin-5-yl)cyclopenten-1-yl]benzenesulfonamide;

l7) ethyl 2-[4-(4-fluorophenyl)-5-[4-(methylsulfonyl) phenyl]oxazol-2-yl]-2-benzyl-acetate;

l8) 2-[4-(4-fluorophenyl)-5-[4-(methylsulfonyl)phenyl]oxazol-2-yl]acetic acid;

l9) 2-(tert-butyl)-4-(4-fluorophenyl)-5-[4-(methylsulfonyl)phenyl]oxazole;

l10) 4-(4-fluorophenyl)-5-[4-(methylsulfonyl)phenyl]-2-phenyloxazole;

m1) 4-(4-fluorophenyl)-2-methyl-5-[4-(methylsulfonyl)phenyl]oxazole; and

m2) 4-[5-(3-fluoro-4-methoxyphenyl)-2-trifluoromethyl-4-oxazolyl]benzenesulfonamide.

m3) 6-chloro-2-trifluoromethyl-2H-1-benzopyran-3-carboxylic acid;

m4) 6-chloro-7-methyl-2-trifluoromethyl-2H-1-benzopyran-3-carboxylic acid;

m5) 8-(1-methylethyl)-2-trifluoromethyl-2H-1-benzopyran-3-carboxylic acid;

m6) 6-chloro-7-(1,1-dimethylethyl)-2-trifluoromethyl-2H-1-benzopyran-3-carboxylic acid;

m7) 6-chloro-8-(1-methylethyl)-2-trifluoromethyl-2H-1-benzopyran-3-carboxylic acid;

m8) 2-trifluoromethyl-3H-naphthopyran-3-carboxylic acid;

m9) 7-(1,1 -dimethylethyl)-2-trifluoromethyl-2H-1-benzopyran-3-carboxylic acid;

m10) 6-bromo-2-trifluoromethyl-2H-1-benzopyran-3-carboxylic acid;

n1) 8-chloro-2-trifluoromethyl-2H-1-benzopyran-3-carboxylic acid;

n2) 6-trifluoromethoxy-2-trifluoromethyl-2H-1-benzopyran-3-carboxylic acid;

n3) 5,7-dichloro-2-trifluoromethyl-2H-1-benzopyran-3-carboxylic acid;

n4) 8-phenyl-2-trifluoromethyl-2H-1-benzopyran-3-carboxylic acid;

n5) 7,8-dimethyl-2-trifluoromethyl-2H-1-benzopyran-3-carboxylic acid;

n6) 6,8-bis(dimethylethyl)-2-trifluoromethyl-2H-1-benzopyran-3-carboxylic acid;

n7) 7-(1-methylethyl)-2-trifluoromethyl-2H-1-benzopyran-3-carboxylic acid;

n8) 7-phenyl-2-trifluoromethyl-2H-1-benzopyran-3-carboxylic acid;

n9) 6-chloro-7-ethyl-2-trifluoromethyl-2H-1-benzopyran-3-carboxylic acid;

n10) 6-chloro-8-ethyl-2-trifluoromethyl-2H-1-benzopyran-3-carboxylic acid;

o1) 6-chloro-7-phenyl-2-trifluoromethyl-2H-1-benzopyran-3-carboxylic acid;

o2) 6,7-dichloro-2-trifluoromethyl-2H-1-benzopyran-3-carboxylic acid;

o3) 6,8-dichloro-2-trifluoromethyl-2H-1-benzopyran-3-carboxylic acid;

o4) 2-trifluoromethyl-3H-naptho[2,1-b]pyran-3-carboxylic acid;

o5) 6-chloro-8-methyl-2-trifluoromethyl-2H-1-benzopyran-3-carboxylic acid;

o6) 8-chloro-6-methyl-2-trifluoromethyl-2H-1-benzopyran-3-carboxylic acid;

o7) 8-chloro-6-methoxy-2-trifluoromethyl-2H-1-benzopyran-3-carboxylic acid;

o8) 6-bromo-8-chloro-2-trifluoromethyl-2H-1-benzopyran-3-carboxylic acid;

o9) 8-bromo-6-fluoro-2-trifluoromethyl-2H-1-benzopyran-3-carboxylic acid;

o10) 8-bromo-6-methyl-2-trifluoromethyl-2H-1-benzopyran-3-carboxylic acid;

p1) 8-bromo-5-fluoro-2-trifluoromethyl-2H-1-benzopyran-3-carboxylic acid;

p2) 6-chloro-8-fluoro-2-trifluoromethyl-2H-1-benzopyran-3-carboxylic acid;

p3) 6-bromo-8-methoxy-2-trifluoromethyl-2H-1-benzopyran-3-carboxylic acid;

p4) 6-[[phenylmethyl)amino]sulfonyl]-2-trifluoromethyl-2H-1-benzopyran-3-carboxylic acid;

p5) 6-[(dimethylamino)sulfonyl]-2-trifluoromethyl-2H-1-benzopyran-3-carboxylic acid;

p6) 6-[(methylamino)sulfonyl]-2-trifluoromethyl-2H-1-benzopyran-3-carboxylic acid;

p7) 6-[(4-morpholino)sulfonyl]-2-trifluoromethyl-2H-1-benzopyran-3-carboxylic acid;

p8) 6-[(1,1-dimethylethyl)aminosulfonyl]-2-trifluoromethyl-2H-1-benzopyran-3-carboxylic acid;

p9) 6-[(2-methylpropyl)aminosulfonyl]-2-trifluoromethyl-2H-1-benzopyran-3-carboxylic acid;

p10) 6-methylsulfonyl-2-trifluoromethyl-2H-1-benzopyran-3-carboxylic acid;

q1) 8-chloro-6-[[(phenylmethyl)amino]sulfonyl]-2-trifluoromethyl-2H-1-benzopyran-3-carboxylic acid;

q2) 6-phenylacetyl-2-trifluoromethyl-2H-1-benzopyran-3-carboxylic acid;

q3) 6,8-dibromo-2-trifluoromethyl-2H-1-benzopyran-3-carboxylic acid;

q4) 8-chloro-5,6-dimethyl-2-trifluoromethyl-2H-1-benzopyran-3-carboxylic acid;

q5) 6,8-dichloro-(S)-2-trifluoromethyl-2H-1-benzopyran-3-carboxylic acid;

q6) 6-benzylsulfonyl-2-trifluoromethyl-2H-1-benzopyran-3-carboxylic acid;

q7) 6-[[N-(2-furylmethyl)amino]sulfonyl]-2-trifluoromethyl-2H-1-benzopyran-3-carboxylic acid;

q8) 6-[[N-(2-phenylethyl)amino]sulfonyl]-2-trifluoromethyl-2H-1-benzopyran-3-carboxylic acid;

q9) 6-iodo-2-trifluoromethyl-2H-1-benzopyran-3-carboxylic acid;

q10) 7-(1,1-dimethylethyl)-2-pentafluoroethyl-2H-1-benzopyran-3-carboxylic acid;

r1) 5,5-dimethyl-3-(3-fluorophenyl)-4-(4-methyl-sulphonyl-2(5H)-fluranone;

r2) 6-chloro-2-trifluoromethyl-2H-1-benzothiopyran-3-carboxylic acid;

r3) 4-[5-(4-chlorophenyl)-3-(trifluoromethyl)-1H-pyrazol-1-yl]benzenesulfonamide;

r4) 4-[5-(4-methylphenyl)-3-(trifluoromethyl)-1H-pyrazol-1-yl]benzenesulfonamide;

r5) 4-[5-(3-fluoro-4-methoxyphenyl)-3-(difluoromethyl)-1H-pyrazol-1-yl]benzenesulfonamide;

r6) 3-[1-[4-(methylsulfonyl)phenyl]-4-trifluoromethyl-1H-imidazol-2-yl]pyridine;

r7) 2-methyl-5-[1-[4-(methylsulfonyl)phenyl]-4-trifluoromethyl-1H-imidazol-2-yl]pyridine;

r8) 4-[2-(5-methylpyridin-3-yl)-4-(trifluoromethyl)-1H-imidazol-1-yl]benzenesulfonamide;

r9) 4-[5-methyl-3-phenylisoxazol-4-yl]benzenesulfonamide;

r10) 4-[5-hydroxymethyl-3-phenylisoxazol-4-yl]benzenesulfonamide;

s1) [2-trifluoromethyl-5-(3,4-difluorophenyl)-4-oxazolyl]benzenesulfonamide;

s2) 4-[2-methyl-4-phenyl-5-oxazolyl]benzenesulfonamide; or

s3) 4-[5-(3-fluoro-4-methoxyphenyl-2-trifluoromethyl)-4-oxazolyl]benzenesulfonamide;

or a pharmaceutically acceptable salt or prodrug thereof.

In a further preferred embodiment of the invention the cyclooxygenase inhibitor can be selected from the class of tricyclic cyclooxygenase-2 selective inhibitors represented by the general structure of Formula IV:

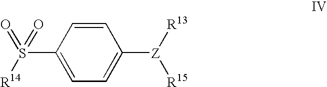

wherein:

Z is selected from the group consisting of partially unsaturated or unsaturated heterocyclyl and partially unsaturated or unsaturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is selected from the group consisting of methyl or amino; and

R¹⁵ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the cyclooxygenase-2 selective inhibitor represented by the above Formula IV is selected from the group of compounds, illustrated in Table 4, which includes celecoxib (B-18), valdecoxib (B-19), deracoxib (B-20), rofecoxib (B-21), etoricoxib (MK-663; B-22), JTE-522 (B-23), or a prodrug thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 4
Examples of Tricyclic COX-2 Selective Inhibitors as Embodiments

| Compound | |
|---|---|
| Number | Structural Formula |
| B-18 | 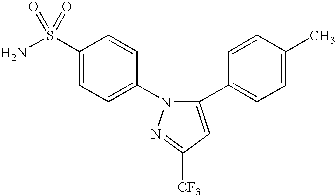 |
| B-19 | 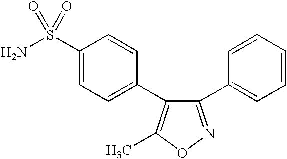 |
| B-20 | 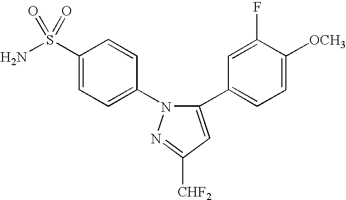 |
| B-21 | 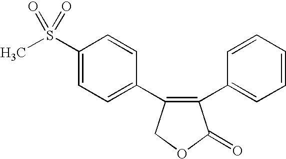 |
| B-22 | 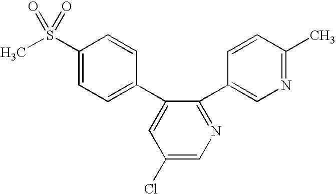 |
| B-23 | 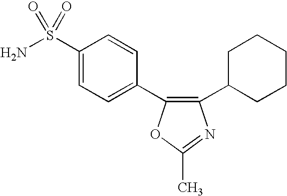 |

In a more preferred embodiment of the invention, the Cox-2 selective inhibitor is selected from the group consisting of celecoxib, rofecoxib and etoricoxib.

In a preferred embodiment of the invention, parecoxib (See, e.g. U.S. Pat. No. 5,932,598), having the structure shown in B-24, which is a therapeutically effective prodrug of the tricyclic cyclooxygenase-2 selective inhibitor valdecoxib, B-19, (See, e.g., U.S. Pat. No. 5,633,272), may be advantageously employed as a source of a cyclooxygenase inhibitor.

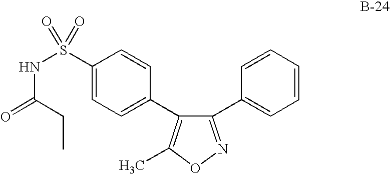

A preferred form of parecoxib is sodium parecoxib.

In another preferred embodiment of the invention, the compound ABT-963 having the formula B-25 that has been previously described in International Publication number WO 00/24719, is another tricyclic cyclooxygenase-2 selective inhibitor which may be advantageously employed.

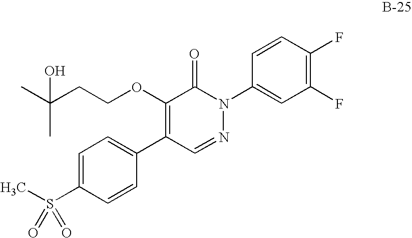

In a further preferred embodiment of the invention the cyclooxygenase inhibitor can be selected from the class of phenylacetic acid derivative cyclooxygenase-2 selective inhibitors represented by the general structure of Formula V:

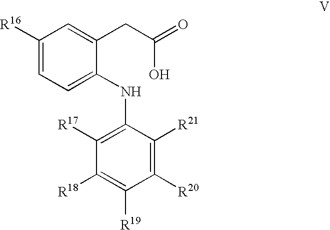

wherein R¹⁶ is methyl or ethyl;

R¹⁷ is chloro or fluoro;

R¹⁸ is hydrogen or fluoro

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl,

provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor that is described in WO 99/11605 is a compound that has the designation of COX189 (CAS RN 346670-74-4), and that has the structure shown in Formula V,

wherein R¹⁶ is ethyl;

R¹⁷ and R¹⁹ are chloro;

R¹⁸ and R²⁰ are hydrogen; and

and R²¹ is methyl.

Compounds that have a structure similar to that shown in Formula V, which can serve as the Cox-2 selective inhibitor of the present invention, are described in U.S. Pat. Nos. 6,310,099 and 6,291,523.

Other preferred cyclooxygenase-2 selective inhibitors that can be used in the present invention have the general structure shown in formula VI, where the J group is a carbocycle or a heterocycle. Particularly preferred embodiments have the structure:

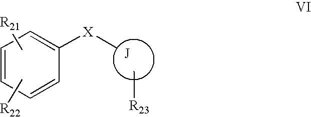

where:

X is O; J is 1-phenyl; R₂₁ is 2-NHSO₂CH₃; R₂₂ is 4-NO₂; and there is no R₂₃ group, (nimesulide), and

X is O; J is 1-oxo-inden-5-yl; R₂₁ is 2-F; R₂₂ is 4-F; and R₂₃ is 6-NHSO₂CH₃, (flosulide); and

X is O; J is cyclohexyl; R₂₁ is 2-NHSO₂CH₃; R₂₂is 5-NO₂; and there is no R₂₃ group, (NS-398); and

X is S; J is 1-oxo-inden-5-yl; R₂₁ is 2-F; R₂₂ F₂ is 4-F; and R₂₃ is 6-N⁻SO₂CH₃.Na⁺, (L-745337); and

X is S; J is thiophen-2-yl; R₂₁ is 4-F; there is no R₂₂ group; and R₂₃ is 5-NHSO₂CH₃, (RWJ-63556); and

X is O; J is 2-oxo-5(R)-methyl-5-(2,2,2-trifluoroethyl)furan-(5H)-3-yl; R₂₁ F, is 3-F; R₂₂ is 4-F; and R₂₃ is 4-(p-SO₂CH₃)C₆H₄, (L-784512).

Further information on the applications of N-(2-cyclohexyloxynitrophenyl)methane sulfonamide (NS-398, CAS RN 123653-11-2), having a structure as shown in formula B-26, have been described by, for example, Yoshimi, N. et al., in Japanese J. Cancer Res., 90(4):406 - 412 (1999); Falgueyret, J. -P. et al., in Science Spectra, available at: http://www.gbhap.com/Science-_Spectra/20-1-article.htm (Jun. 6, 2001); and Iwata, K. et al., in Jpn. J. Pharmacol., 75(2):191-194 (1997).

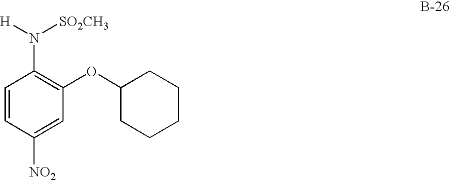

An evaluation of the antiinflammatory activity of the cyclooxygenase-2 selective inhibitor, RWJ 63556, in a canine model of inflammation, was described by Kirchner et al., in J Pharmacol Exp Ther 282, 1094-1101 (1997).

Other materials that can serve as he cyclooxygenase-2 selective inhibitor of the present invention include diarylmethylidenefuran derivatives that are described in U.S. Pat. No. 6,180,651. Such diarylmethylidenefuran derivatives have the general formula shown below in formula VII:

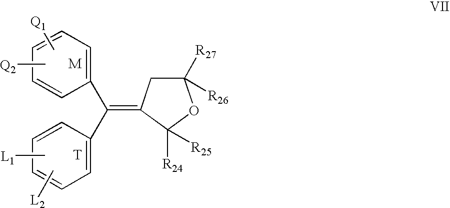

wherein:

the rings T and M independently are:

a phenyl radical,

a naphthyl radical,

a radical derived from a heterocycle comprising 5 to 6 members and possessing from 1 to 4 heteroatoms, or

a radical derived from a saturated hydrocarbon ring having from 3 to 7 carbon atoms;

at least one of the substituents Q₁, Q₂, L₁ or L₂ is:

an —S(O)ₙ—R group, in which n is an integer equal to 0, 1 or 2 and R is a lower alkyl radical having 1 to 6 carbon atoms or a lower haloalkyl radical having 1 to 6 carbon atoms, or an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q₁ and Q₂ or L₁ and L₂ are a methylenedioxy group; and

R₂₄, R₂₅, R₂₆ and R₂₇ independently are:

a hydrogen atom,

a halogen atom,

a lower alkyl radical having 1 to 6 carbon atoms,

a lower haloalkyl radical having 1 to 6 carbon atoms, or

an aromatic radical selected from the group consisting of phenyl, naphthyl, thienyl, furyl and pyridyl; or,

R₂₄, R₂₅ or R₂₆, R₂₇ are an oxygen atom, or

R₂₄, R₂₅ or R₂₆, R₂₇, together with the carbon atom to which they are attached, form a saturated hydrocarbon ring having from 3 to 7 carbon atoms;

or an isomer or prodrug thereof.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Cyclooxygenase-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs.com/NEWS/Inflam 1.htm, Oct. 4, 2001, where it was reported that S-33516 is a tetrahydroisoinde derivative which has IC50 values of 0.1 and 0.001 mM against cyclooxygenase-1 and cyclooxygenase-2, respectively. In human whole blood, S-33516 was reported to have an ED₅₀=0.39 mg/kg.

Cox-2 selective inhibitors that are useful in the subject method and compositions can include the compounds that are described in U.S. Pat. Nos. 6,310,079; 6,306,890 and 6,303,628 (bicycliccarbonyl indoles); U.S. Pat. Nos. 6,133,292; 6,020,343; 5,981,576 ((methylsulfonyl)phenyl furanones); U.S. Pat. No. 6,083,969 (diarylcycloalkano and cycloalkeno pyrazoles); U.S. Pat. No. 6,077,869 (aryl phenylhydrazines); U.S. Pat. No. 6,071,936 (substituted pyridines); U.S. Pat. No. 6,307047 (pyridazinone compounds); U.S. Pat. No. 6,140,515 (3-aryl-4-aryloxyfuran-5-ones); and U.S. Pat. Nos. 6,002,014; 5,994,381; and 5,945,539 (oxazole derivatives).

Cyclooxygenase-2 selective inhibitors that are useful in the present invention can be supplied by any source as long as the cyclooxygenase-2-selective inhibitor is pharmaceutically acceptable. Cyclooxygenase-2-selective inhibitors can be isolated and purified from natural sources or can be synthesized. Cyclooxygenase-2-selective inhibitors should be of a quality and purity that is conventional in the trade for use in pharmaceutical products.

The cyclooxygenase -2 selective inhibitors of Compound Numbers B-1 through B-25 and naturally occuring COX-2 selective inhibitors are referred to herein collectively as Group II.

Combinations of the present invention are preferably selected from compounds of Group I (n-3 PUFAs) and Group II (COX-2 selective inhibitors). While it is contemplated in the present invention that several compounds from Group I may be combined with one or more compounds of Group II, it is preferred to combine between one and three compounds of Group I with a single compound of Group II.

Exemplary combinations of Group I n-3 PUFAs and COX-2 selective inhibitors are shown in Table 5.

TABLE 5
Exemplary Combinations

| Example Number | Compound I (Group I) | Compound II (Group II) |
|---|---|---|
| 1 | A-1 | B-1 |
| 2 | A-1 | B-2 |
| 3 | A-1 | B-3 |
| 4 | A-1 | B-4 |
| 5 | A-1 | B-5 |
| 6 | A-1 | B-6 |
| 7 | A-1 | B-7 |
| 8 | A-1 | B-8 |
| 9 | A-1 | B-9 |
| 10 | A-1 | B-10 |
| 11 | A-1 | B-11 |
| 12 | A-1 | B-12 |
| 13 | A-1 | B-13 |
| 14 | A-1 | B-14 |
| 15 | A-1 | B-15 |
| 16 | A-1 | B-16 |
| 17 | A-1 | B-17 |
| 18 | A-1 | B-18 |
| 19 | A-1 | B-19 |
| 20 | A-1 | B-20 |
| 21 | A-1 | B-21 |
| 22 | A-1 | B-22 |
| 23 | A-1 | B-23 |
| 24 | A-1 | B-24 |
| 25 | A-1 | B-25 |
| 26 | A-2 | B-1 |
| 27 | A-2 | B-2 |
| 28 | A-2 | B-3 |
| 29 | A-2 | B-4 |
| 30 | A-2 | B-5 |
| 31 | A-2 | B-6 |
| 32 | A-2 | B-7 |
| 33 | A-2 | B-8 |
| 34 | A-2 | B-9 |
| 35 | A-2 | B-10 |
| 36 | A-2 | B-11 |
| 37 | A-2 | B-12 |
| 38 | A-2 | B-13 |
| 39 | A-2 | B-14 |
| 40 | A-2 | B-15 |
| 41 | A-2 | B-16 |
| 42 | A-2 | B-17 |
| 43 | A-2 | B-18 |
| 44 | A-2 | B-19 |
| 45 | A-2 | B-20 |
| 46 | A-2 | B-21 |
| 47 | A-2 | B-22 |
| 48 | A-2 | B-23 |
| 49 | A-2 | B-24 |
| 50 | A-2 | B-25 |
| 51 | A-3 | B-1 |
| 52 | A-3 | B-2 |
| 53 | A-3 | B-3 |
| 54 | A-3 | B-4 |
| 55 | A-3 | B-5 |
| 56 | A-3 | B-6 |
| 57 | A-3 | B-7 |
| 58 | A-3 | B-8 |
| 59 | A-3 | B-9 |
| 60 | A-3 | B-10 |
| 61 | A-3 | B-11 |
| 62 | A-3 | B-12 |
| 63 | A-3 | B-13 |
| 64 | A-3 | B-14 |
| 65 | A-3 | B-15 |
| 66 | A-3 | B-16 |
| 67 | A-3 | B-17 |
| 68 | A-3 | B-18 |
| 69 | A-3 | B-19 |
| 70 | A-3 | B-20 |
| 71 | A-3 | B-21 |
| 72 | A-3 | B-22 |
| 73 | A-3 | B-23 |
| 74 | A-3 | B-24 |
| 75 | A-3 | B-25 |
| 76 | A-4 | B-1 |
| 77 | A-4 | B-2 |
| 78 | A-4 | B-3 |
| 79 | A-4 | B-4 |
| 80 | A-4 | B-5 |
| 81 | A-4 | B-6 |
| 82 | A-4 | B-7 |
| 83 | A-4 | B-8 |
| 84 | A-4 | B-9 |
| 85 | A-4 | B-10 |
| 86 | A-4 | B-11 |
| 87 | A-4 | B-12 |
| 88 | A-4 | B-13 |
| 89 | A-4 | B-14 |
| 90 | A-4 | B-15 |
| 91 | A-4 | B-16 |
| 92 | A-4 | B-17 |
| 93 | A-4 | B-18 |
| 94 | A-4 | B-19 |
| 95 | A-4 | B-20 |
| 96 | A-4 | B-21 |
| 97 | A-4 | B-22 |
| 98 | A-4 | B-23 |
| 99 | A-4 | B-24 |
| 100 | A-4 | B-25 |
| 101 | A-5 | B-1 |
| 102 | A-5 | B-2 |
| 103 | A-5 | B-3 |
| 104 | A-5 | B-4 |
| 105 | A-5 | B-5 |
| 106 | A-5 | B-6 |
| 107 | A-5 | B-7 |
| 108 | A-5 | B-8 |
| 109 | A-5 | B-9 |
| 110 | A-5 | B-10 |
| 111 | A-5 | B-11 |
| 112 | A-5 | B-12 |
| 113 | A-5 | B-13 |
| 114 | A-5 | B-14 |
| 115 | A-5 | B-15 |
| 116 | A-5 | B-16 |
| 117 | A-5 | B-17 |
| 118 | A-5 | B-18 |
| 119 | A-5 | B-19 |
| 120 | A-5 | B-20 |
| 121 | A-5 | B-21 |
| 122 | A-5 | B-22 |
| 123 | A-5 | B-23 |
| 124 | A-5 | B-24 |
| 125 | A-5 | B-25 |
| 126 | A-6 | B-1 |
| 127 | A-6 | B-2 |
| 128 | A-6 | B-3 |
| 129 | A-6 | B-4 |
| 130 | A-6 | B-5 |
| 131 | A-6 | B-6 |
| 132 | A-6 | B-7 |
| 133 | A-6 | B-8 |
| 134 | A-6 | B-9 |
| 135 | A-6 | B-10 |
| 136 | A-6 | B-11 |
| 137 | A-6 | B-12 |
| 138 | A-6 | B-13 |
| 139 | A-6 | B-14 |
| 140 | A-6 | B-15 |
| 141 | A-6 | B-16 |
| 142 | A-6 | B-17 |
| 143 | A-6 | B-18 |
| 144 | A-6 | B-19 |
| 145 | A-6 | B-20 |
| 146 | A-6 | B-21 |
| 147 | A-6 | B-22 |
| 148 | A-6 | B-23 |
| 149 | A-6 | B-24 |
| 150 | A-6 | B-25 |

These preferred combinations are for illustrative purposes only, and are not intended to be limiting in scope.

In addition, it may be desirable to combine more than one n-3 PUFA with a COX-2 selective inhibitor. For example, in one embodiment of the present invention, the n-3 PUFA is a combination of 18:3 ω-3, α-linolenic acid (ALA) and 18:4 ω-3, stearidonic acid (SDA), formula A-2. In another embodiment of the present invention, the n-3 PUFA is a combination of 18:3 ω-3, α-linolenic acid (ALA) and 20:4 ω-3, eicosatetraenoic acid, formula A-3. In a further embodiment of the present invention, the n-3 PUFA is a combination of 18:3 ω-3, α-linolenic acid (ALA) and 20:5 ω-3, eicosapentaenoic acid (EPA), formula A-4. In another embodiment of the present invention, the n-3 PUFA is a combination of 18:3 ω-3, α-linolenic acid (ALA) and 22:5 ω-3, docosapentaenoic acid, formula A-5.

In a further embodiment of the present invention, n-3 PUFA is a combination of 18:3 ω-3, α-linolenic acid (ALA) and 22:6 ω-3, docosahexaenoic acid (DHA), formula A-6. In a further embodiment of the present invention, n-3 PUFA is a combination of 18:4 ω-3, stearidonic acid (SDA), formula A-2 and 20:4 ω-3, eicosatetraenoic acid, formula A-3. In another embodiment of the present invention, the n-3 PUFA is a combination of 18:4 ω-3, stearidonic acid (SDA), formula A-2 and 20:5 ω-3, eicosapentaenoic acid (EPA), formula A-4. In a further embodiment of the present invention, the n-3 PUFA is a combination of 18:4 ω-3, stearidonic acid (SDA), formula A-2 and 22:5 ω-3, docosapentaenoic acid, formula A-5. In another embodiment of the present invention, the n-3 PUFA is a combination of 18:4 ω-3, stearidonic acid (SDA), formula A-2 and 22:6 ω-3, docosahexaenoic acid (DHA), formula A-6. In a further embodiment of the present invention, the n-3 PUFA is a combination of 20:4 ω-3, eicosatetraenoic acid, formula A-3 and eicosapentaenoic acid (EPA), formula A-4. In another embodiment of the present invention, the n-3 PUFA is a combination of 20:4 ω-3, eicosatetraenoic acid, formula A-3 and 22:5 ω-3, docosapentaenoic acid, formula A-5. In another embodiment of the present invention, the n-3 PUFA is a combination of 20:4 ω-3, eicosatetraenoic acid, formula A-3 and 22:6 ω-3, docosahexaenoic acid (DHA), formula A-6. In a further embodiment of the present invention, the n-3 PUFA is a combination of 20:5 ω-3, eicosapentaenoic acid (EPA), formula A-4 and 22:5 ω-3, docosapentaenoic acid, formula A-5. In another embodiment of the present invention, the n-3 PUFA is a combination of eicosapentaenoic acid (EPA), formula A-4 and 22:6 ω-3, docosahexaenoic acid (DHA), formula A-6. In a further embodiment of the present invention, the n-3 PUFA is a combination of 22:5 ω-3, docosapentaenoic acid, formula A-5 and 22:6 ω-3, docosahexaenoic acid (DHA), formula A-6. In another embodiment of the present invention, the n-3 PUFA is a combination of 18:3 ω-3, α-linolenic acid (ALA), 18:4 ω-3, stearidonic acid (SDA), formula A-2 and 20:4 ω-3, eicosatetraenoic acid, formula A-3. These examples are merely illustrative, and other combinations will occur to those skilled in the art.

Although the combination of the present invention may include administration of an n-3 PUFA component and a cyclooxygenase-2 selective inhibitor component within an effective time of each respective component, it is preferable to administer both respective components contemporaneously, and more preferable to administer both respective components in a single delivery dose. In particular, the combinations of the present invention can be administered:

A) Orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The said aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspension may be formulated by suspending the active ingredients in an omega-3 fatty acid of the present invention, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavouring agents may be added to provide a palatable oral preparation. These compositions may be preserved by the addition of an antioxidant such as ascorbic acid.

Dispersible powders and granules suitable for peparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase is preferably the n-3 polyunsaturated fatty acid, for example canola oil containing SDA, but may also be another, non-omega-3 vegetable oil, for example olive oil or arachis oils, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan mono-oleate, and condensation products of the said partial esters with ethylene oxide, for example polyoxyethylene sorbitan monooleate. The emulsion may also contain sweetening and flavoring agents.

Syrups and elixirs may be formulated with sweetening agents, for example glycerol, sorbitol or sucrose. Such formulations may also contain a demulcent, a preservative and flavoring andcoloring agents;

B) Parenterally, either subcutaneously, or intravenously, or intramuscularly, or intrasternally, or by infusion techniques, in the form of sterile injectable aqueous or olagenous suspensions. This suspension may be formulated according to the known art using those suitable dispersing of wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents

that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, the n-3 polyunsaturated fatty acids of the present invention may find use in the preparation of injectables;

C) By inhalation, in the form of aerosols or solutions for nebulizers;

D) Rectally, in the form of suppositories prepared by mixing the drug with a suitable non-irratating excipient which is solid at ordinary temperature but liquid at the rectal temperature and will therefore melt in the rectum to release the drug. Such materials are cocoa butter and poly-ethylene glycols;

E) Topically, in the form of creams, ointments, jellies, collyriums, solutions or suspensions.

Daily dosages can vary within wide limits and will be adjusted to the individual requirements in each particular case. In general, for administration to adults, an appropriate daily dosage is in the range of about 5 mg to about 500 mg, although the upper limit may be exceeded if expedient. The daily dosage can be administered as a single dosage or in divided dosages.

Preferred delivery systems include capsules, tablets, and gelatin capsules, for example.

The dose or effective amount to be administered to a patient and the frequency of administration to the subject which is readily determined by one or ordinary skill in the art, by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

Those skilled in the art will appreciate that dosages may also be determined with guidance from Goodman & Goldman's The Pharmacological Basis of Therapeutics, Ninth Edition (1996), Appendix II, pp. 1707-1711.

Effective amounts of n-3 PUFAs are in the range of 0.1-10 grams (g) per day, preferably between 0.5 and 7 grams per day, and most preferably between 1 and 5 grams per day. Several nationalities with the present exception of the United States of America have established recommended daily intakes (RDI's) of n-3 PUFAs, and these RDIs may be useful in determining an appropriate daily dosage. Of course, the effective amount depends in part on the type of n-3 PUFA used, such that if α linolenic acid is used, the upper range of daily dosage is preferred, and if SDA or DHA or EPA is used, a smaller daily dosage is preferred. Additionally, if the n-3 PUFA is derived from fish oil, lower dosages are preferred to avoid excessive ingestion of vitamins A and D, which could be toxic at dosages that would yield over 5 grams of n-3 PUFAs per day.

Typically, the amount of α-linolenic acid metabolite administered will be between about 1 mg/Kg/day and about 300 mg/Kg/day. Preferably, the amount of the metabolite administered is between about 10 mg/Kg/day and about 150 mg/Kg/day. The desired dosage may be administered as most efficacious, generally from 1-5 doses per day, desirably from 1-3 doses per day. Preferably, the α-linolenic acid metabolite administered to the mammal is SDA or a combination of SDA and at least one other ALA metabolite.

The pharmaceutical compositions may contain a cyclooxygenase-2 selective inhibitor in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight and most preferably from about 1 to 20 mg/kg body weight, may be appropriate. The daily dose can be administered in one to four doses per day.

Several animal models are available which are appropriate for evaluation of prevention of cardiovascular conditions including the prevention of atherosclerosis. See Stehbens, Prog. Card. Dis., XXIX, 1007-28 (1986) and Zhang et al., Science, 258, 468-71 (1992).

An APoE mouse model for atherosclerosis has been described by Roselear et al. (Arterioscle. Thromb. Vasc. Biol., 16, 1013-18 (1996)). The cyclooxygenasse-2 inhibitor should be active, at a dose of 20 mg/kg, in preventing atherosclerotic lesions.

The examples which follow are intended to illustrate certain preferred embodiments of the n-3 PUFAs of the present invention, and no limitation of the invention is implied. The n-3 PUFAs used in all of the following examples are in the free acid form (100% pure) when used in cell culture and in the ethyl ester form (>85% pure) when administered in vivo. The ethyl esters of stearidonic acid (SDA-EE), eicosapentaenoic acid (EPA-EE) and docosahexaenoic acid (DHA-EE) are derived from fish oil, and are available from KD Pharma (Bexbach, Germany). The ethyl ester of stearidonic acid may be further purified by Callanish, Ltd. (Scotland, U.K.) to increase the SDA-EE content from approximately 60% to 85% and also to decrease the EPA-EE content from approximately 8% to 0.2%. The ethyl esters of α-linolenic acid (ALA-EE) and γ-linolenic acid (GLA-EE), which are derived from plant oils, are at least 95% pure and may be purchased from Callanish, Ltd. Administration of the fatty acid ethyl esters in rodents is scaled allometrically by caloric equivalency to reflect the human equivalent amount of fatty acid consumed per day (=g/day human equivalent dose).

### EXAMPLE 1

### The US17 Diet

In order to study the effects of PUFAs on cardiovascular disease and colon tumor formation and promotion in rodents, a diet (the “US17 diet”) is designed to mimic the human western diet. The human western diet contains high levels of saturated fatty acids and linoleic acid, both of which have been linked to cardiovascular disease and cancer formation. The components of the US 17 diet are set forth in Tables 1-6, below.

TABLE 1
Ingredients of the US17 diet.

| Ingredient | Amount (grams) |
|---|---|
| Casein, Alcx | 200 |
| L-Cystine | 3 |
| Corn Starch | 240 |
| Maltodextrin 10 | 75 |
| Sucrose | 100 |
| Cellulose | 50 |
| Cocoa Butter (Deodorized) | 37.5 |
| Linseed Oil | 4.5 |
| Palm Oil (Bleached, deodorized) | 52.5 |
| Safflower Oil, USP | 28.5 |
| Sunflower Oil, Trisum Extra | 27 |
| t-BHQ | 0.03 |
| Salts (See Table 2) | 10 |
| Dicalcium Phosphate | 13 |
| Calcium carbonate | 5.5 |
| Potassium citrate (Monohydrate) | 16.5 |
| Vitamins (See Table 3) | 10 |
| Choline bitartrate | 2 |
| α-Vitamin E acetate (500 IU/gm) | 0.13 |
| Total (grams) | 875.16 |

TABLE 2
Salt mixture of the US17 diet

| Ingredient | Amount (gm) |
|---|---|
| Sodium Chloride | 25.90 |
| Magnesium Oxide | 4.19 |
| Magnesium Sulfate ?7H₂O | 25.76 |
| Chromium Potassium Sulfate ?12H₂O | 0.19 |
| Cupric Carbonate | 0.10 |
| Sodium Fluoride | 0.02 |
| Potassium Iodate | 0.003 |
| Ferric Citrate | 2.10 |
| Manganous Carbonate | 1.23 |
| Ammonium Molybdate ?4H₂O | 0.03 |
| Sodium Selenite | 0.003 |
| Zinc Carbonate | 0.56 |
| Sucrose | 39.91 |
| Total | 100 |

TABLE 3
Vitamin mixture of the US17 diet

| Ingredient | Amount (gm) |
|---|---|
| Vitamin A Palmitate | 0.08 |
| 500,000 IU/gm | |
| Vitamin D3 | 0.10 |
| 100,000 IU/gm | |
| Vitamin E Acetate | 1.00 |
| 500 IU/gm | |
| Menadione Sodium Bisulfite | 0.008 |
| Biotin 1.0% | 0.20 |
| Cyanocobalamin 0.1% | 0.10 |
| Folic Acid | 0.02 |
| Nicotinic Acid | 0.30 |
| Calcium Pantothenate | 0.16 |
| Pyridoxine-HCl | 0.07 |
| Riboflavin | 0.06 |
| Thiamin HCl | 0.06 |
| Sucrose | 97.84 |
| Total | 100 |

TABLE 4
Fatty acid content of the US17 diet

| | |
|---|---|
| Fatty Acid | Amount |

| | | |
|---|---|---|
| C14, Myristic | 0.7 | gms |
| C16, Palmitic | 34.6 | gms |
| C16:1, Palmitoleic | 0.2 | gms |
| C18, Stearic | 17.5 | gms |
| C18:1, Oleic | 60.5 | gms |
| C18:2, Linoleic | 30.2 | gms |
| C18:3, Linolenic | 3.1 | gms |
| C20, Arachidic | 0.4 | gms |
| Saturated | 36.1 | weight % |
| Monounsaturated | 41.3 | weight % |
| Polyunsaturated | 22.6 | weight % |

TABLE 5
Nutritional content of the US17 diet

| | |
|---|---|
| Nutrient | Amount |

| | | |
|---|---|---|
| Protein | 22.7 | weight % |
| Carbohydrate | 48.6 | weight % |
| Fat | 17.1 | weight % |
| Fiber | 5.7 | weight % |
| Protein | 20.7 | kcal % |
| Carbohydrate | 44.2 | kcal % |
| Fat | 35.1 | kcal % |

TABLE 6
Comparison between rodent US17 diet and
human, western diet

Kcal %

| | Rodent diet | |
|---|---|---|
| Nutrient | (US17) | Human Diet |
| Protein | 21 | 15 |
| Carbohydrate | 44 | 50 |
| Fat | 35 | 35 |
| Fatty Acid Composition | | |
| <C16 | 0.2 | 1.6 |
| 16:0 | 8.6 | 7.9 |
| 18:0 | 4.3 | 3.9 |
| 18:1 n-9 (oleic acid | 14.6 | 14.0 |
| cassette) | | |
| 18:2 n-6 | 7.0 | 6.9 |
| 18:3 n-3 | 0.7 | 0.7 |
| n-6:n-3 ratio | 10:1 | 10:1 |

The fatty acid test agent is substituted for oleic acid (=oleic acid cassette) and the dose, when scaled allometrically based upon Kcal (energy) %, is in the range readily consumed by humans (i.e., 0.1 to 10 g/day human equivalent dose).

### EXAMPLE 2

### Uptake of ¹⁴C-ALA, ¹⁴C-SDA and ¹⁴C-EPA by HepG2 Cells

The uptake of stearidonic acid by HepG2 cells is compared to that of α-linolenic acid and eicosapentaenoic acid.

To a culture medium containing HepG2 cells is added 20 μM ¹⁴C-ALA, ¹⁴C-SDA or ¹⁴C-EPA complexed to fatty acid free BSA. The amount of ¹⁴C-ALA, ¹⁴C-SDA or ¹⁴C-EPA taken up by the HepG2 cells is measured at 6 hours, 24 hours and 48 hours after addition of the fatty acid. A scintillation counter is used to measure the total amount of radioactivity in the HepG2 cells and the amount remaining in the medium.

¹⁴C-ALA, ¹⁴C-SDA and ¹⁴C-EPA are taken up equally by HepG2 cells. Approximately 95% of each radiolabeled fatty acid is taken up by the cells within the first six hours of incubation.

### EXAMPLE 3

### Metabolism of Stearidonic Acid to Long Chain n-3 Polyunsaturated Fatty Acids in HepG2 Cells

The metabolism of stearidonic acid to long chain n-3 PUFAs (eicosatetraenoic acid (20:4n-3), eicosapentaenoic acid (20:5n-3), docosapentaenoic acid (22:5n-3), and docosahexaenoic acid (22:6n-3)) in HepG2 cells is compared to that of α-linolenic acid. HepG2 cells are allowed to take up ¹⁴C-ALA or ¹⁴C-SDA as described in Example 2. The total amount of ¹⁴C-EPA, ¹⁴C-DPA and ¹⁴C-DHA present in the HepG2 cells is measured at 6 hours, 24 hours and 48 hours after addition of the fatty acid by argentation thin layer chromatography (TLC). The amount of each fatty acid present as a band on the TLC plate is quantified by electronic autoradiography using an Instant Imager available from Packard (Meriden, Conn.).

The metabolism of SDA in Hep2G cells to long chain n-3 PUFAs is faster than that of ALA. Nearly 95% of the ¹⁴C-SDA is metabolized to ¹⁴C-fatty acid end products (i.e., EPA or DHA) or ¹⁴C fatty acid intermediates (i.e., 20:4 n-3, 22:5 n-3 and 24:5 n-3). ¹⁴C-SDA is metabolized more efficiently to ¹⁴C-EPA than is ¹⁴C-ALA (55% versus 24%).

### EXAMPLE 4

### Metabolism of Stearidonic Acid to EPA and DHA in HepG2 Cells

The metabolism of stearidonic acid to eicosapentaenoic acid (20:5 n-3) and docosahexaenoic acid (22:6 n-3)) in HepG2 cells is compared to that of α-linolenic acid.

HepG2 cells are allowed to take up ¹⁴C-ALA, ¹⁴C-SDA or ¹⁴C-EPA as described in Example 2. The amount of ¹⁴C-EPA and ¹⁴C-DHA present in the HepG2 cells is measured at 6 hours, 24 hours and 48 hours after addition of the fatty acids by argentation thin layer chromatography (TLC) as described in Example 3. The amount of ¹⁴C-EPA and ¹⁴C-DHA present as bands on the TLC plate are quantified by electronic autoradiography using an Instant Imager as described in Example 3.

Radiolabeled EPA is included as a control to evaluate its maintenance in HepG2 cells over time. SDA is metabolized more efficiently to EPA than is ALA (55% versus 24%). The amount of EPA derived from SDA is actually quite similar to the amount of EPA that remained after incubation with EPA itself (55% versus 63%). SDA is metabolized to DHA more efficiently than is ALA (6% versus 3%). In comparison, approximately 11% of EPA is metabolized to DHA. Overall, the results show that SDA is metabolized to EPA and further to DHA at a rate that is approximately twice that of ALA.

### EXAMPLE 5

### Metabolism of Stearidonic Acid in Mice (Time Course Analysis Using Radiolabeled Fatty Acids)

The metabolism of stearidonic acid to long chain n-3 polyunsaturated fatty acids (i.e., eicosapentaenoic acid (EPA), docosapentaenoic acid (DPA n-3), and docosahexaenoic acid (DHA)) in mouse liver is compared to that of α-linolenic acid, eicosapentaenoic acid and docosahexaenoic acid. The method used is a time course study using ¹⁴C-labeled fatty acids.

Mice are fed the US 17 diet for a period of one month in order to achieve steady-state fatty acid metabolism. After achieving steady-state fatty acid metabolism, the mice are administered an intraperitoneal injection containing 10 μCi of ¹⁴C-ALA, ¹⁴C-SDA, ¹⁴C-EPA or ¹⁴C-DHA. Mice are sacrificed 3 hours, 8 hours or 24 hours post-injection, and the total amount of ¹⁴C-EPA, ¹⁴C-DPA n-3 and ¹⁴C-DHA is measured by argentation thin layer chromatography followed by direct electronic autoradiography, as described in Example 3. The order of metabolism of the PUFAs is DHA=EPA>SDA>ALA at 24 hours. SDA is metabolized to long chain n-3 PUFAs more efficiently than ALA in vivo.

### EXAMPLE 6

### Metabolism of Stearidonic Acid in Mice (End Point Analysis Using Cold Fatty Acid Ethyl Esters)

The metabolism of n-6 and n-3 PUFAs in rats and mice is similar to that of humans. Lands, W. E. M., Morris, A., Libelt, B., Lipids, Vol. 25(9), pp. 505-516 (1990). As such, fatty acid metabolic results from studies with rats and mice would be predicted to be similar in humans.

The metabolism of stearidonic acid to long chain n-3 polyunsaturated fatty acids (i.e., eicosapentaenoic acid (EPA), docosapentaenoic acid (DPA n-3) and docosahexaenoic acid (DHA)) in mouse liver is compared to that of α-linolenic acid, eicosapentaenoic acid and docosahexaenoic acid. The method used is a non-radioactive, dose response, metabolic end-point study.

Mice are fed a US 17 diet containing α-linolenic acid ethyl ester (ALA-EE), stearidonic acid ethyl ester (SDA-EE), eicosapentaenoic acid ethyl ester (EPA-EE), or docosahexaenoic acid ethyl ester (DHA-EE) in an amount equivalent to a human western diet containing 1, 3 or 10 g/day of the fatty acid (g/day human equivalent dose). In order to maintain a 17% fat (37% energy) content in the US17 diet, oleic acid (18:1 n-9), as an oleic acid cassette, is removed from the US17 diet in an amount equal to the amount of fatty acid ester that is added. Oleic acid is selected as the replacement fatty acid because literature reports indicate that oleic acid is neutral with respect to inflammation and cancer.

After one month on the respective US17-based diets, the mice are sacrificed and the fatty acid composition of each of their livers is analyzed by gas chromatography. The sum of the long chain n-3 PUFAs (i.e., EPA+DPA n-3+DHA) increases dose dependently in liver tissue in the following rank order: DHA-EE>EPA-EE>SDA-EE>ALA-EE. These results show that SDA is metabolized to long chain n-3 polyunsaturated fatty acids better than ALA. Each of the dietary n-3 PUFAs decreases the level of arachidonic acid in liver tissue dose dependently. This is significant because arachidonic acid metabolites (e.g., prostaglandins, leukotrienes, and HETEs (hydroxyeicosatetraenoic acid)) are correlated with cardiovascular disease, inflammation, and tumorigenesis. The group “basal” refers to mice that are fed the standard rodent chow diet just prior to switching to diets that are US17 based. The results show that the level of the sum of the long chain n-3 PUFAs or arachidonic acid is the same, indicating that the US17 diet does not significantly alter fatty acid composition compared to the standard rodent chow diet.

### EXAMPLE 7

### Metabolism of Stearidonic Acid in Rats (End Point Analysis Using Cold Fatty Acid Ethyl Esters)

The metabolism of stearidonic acid to long chain n-3 polyunsaturated fatty acids (i.e., eicosapentaenoic acid (EPA), docosapentaenoic acid (DPA n-3) and docosahexaenoic acid (DHA)) in rat liver is compared to that of α-linolenic acid, eicosapentaenoic acid and docosahexaenoic acid. The method used is as described in Example 6 except that rats are used in place of mice.

These results show that SDA is metabolized to long chain n-3 polyunsaturated fatty acids better than ALA. SDA causes a greater decrease in the level of arachidonic acid than does either ALA or EPA.

### EXAMPLE 8

### Effect of n-3 and n-6 PUFAs on Intestinal Cancer in the Min/+ Mouse Model

The efficacy of select n-3 and n-6 polyunsaturated fatty acids is evaluated in the Min/+ mouse model of intestinal cancer. The following fatty acid ethyl esters are tested for their effect on intestinal polyp formation: 1) ALA-EE; 2) SDA-EE; 3) EPA-EE; 4) DHA-EE; 5) GLA-EE (alpha-linolenic adid, 18:3 n-6); and 6) CLA-EE (conjugated linoleic acid; c9t11-18:2 (77%)+c9c11-18:2 (18%)+other isomers (5%)).

These fatty acid ethyl esters are added to the US17 diet to provide 10 g/day human equivalent dose (3% wt.). The NSAID, sulindac (320 ppm), serves as the positive control. In order to maintain a 17% fat (37 en %) content in the US17 diet, oleic acid (18:1 n-9) is removed from the US17 diet in an amount equal to the amount of the fatty acid ethyl ester that is added. Mice are received at approximately five weeks of age and are fed the test diets upon receipt. After seven weeks on the respective test diet, the mice are sacrificed and the intestinal polyps are counted and measured. The results of these analyses are presented in Table 6.

TABLE 6
Effect of various fatty acids on tumor size and number in the large and small intestine of Min/+ mice

| | US17 | ALA | CLA | DHA | EPA | GLA | SDA | Sulindac |
|---|---|---|---|---|---|---|---|---|
| | (n = 10) | (n = 10) | (n = 9) | (n = 10) | (n = 10) | (n = 9) | (n = 10) | (n = 9) |
| Total large in- | 13 (7/10) | 8 (5/10) | 10 (5/9) | 18 (9/10) | 9 (6/10) | 11 (6/9) | 2 (2/10) | 2 (2/9) |
| testine tumors/ | | | | | | | | |
| group | | | | | | | | |
| Avg. large in- | 1.3 ± 0.6^{ab} | 0.8 ± 0.3^{bcd} | 1.1 ± 0.4^{abcd} | 1.8 ± 0.4^{a} | 0.9 ± 0.3^{abcd} | 1.2 ± 0.4^{abc} | 0.2 ± 0.1^{d} | 0.2 ± 0.2^{ad} |
| testine tumors/ | | | | | | | | |
| mouse | | | | | | | | |
| Avg. large in- | 2.96 ± 0.20^{b} | 2.75 ± 0.46^{ab} | 3.03 ± 0.52^{a} | 3.01 ± 0.20^{a} | 2.48 ± 0.39^{ab} | 2.57 ± 0.30^{ab} | 1.50 ± 0.00^{b} | 2.00 ± 0.00^{ab} |
| testine tumor | | | | | | | | |
| size | | | | | | | | |
| Avg. large in- | 3.70 ± 1.51^{ab} | 2.15 ± 0.82^{bcd} | 3.47 ± 1.29^{ab} | 5.06 ± 0.85^{a} | 2.25 ± 0.75^{bcd} | 3.25 ± 1.34^{abc} | 0.30 ± 0.20^{d} | 0.44 ± 0.29^{cd} |
| testine tumor | | | | | | | | |
| load | | | | | | | | |
| Total small in- | 32 | 364 | 40 | 280 | 17 | 42 | 18 | 29 |
| testine tumors/ | | | | | | | | |
| group | | | | | | | | |
| Avg. small in- | 33.7 ± 4.5^{ab} | 36.4 ± 6.3^{ab} | 45.4 ± 8.2^{a} | 28.0 ± 5.6^{bc} | 17.6 ± 2.2^{cd} | 46.8 ± 7.1^{a} | 18.5 ± 1.9^{c} | 3.2 ± 1.1^{d} |
| testine tumors/ | | | | | | | | |
| mouse | | | | | | | | |
| Avg. small in- | 1.32 ± 0.05^{a} | 1.23 ± 0.04^{ab} | 1.24 ± 0.03^{ab} | 1.16 ± 0.04^{ab} | 1.05 ± 0.03^{b} | 1.30 ± 0.04^{ab} | 1.07 ± 0.02^{ab} | 1.25 ± 0.29^{ab} |
| testine tumor | | | | | | | | |
| size (mm) | | | | | | | | |
| Avg. small in- | 43.62 ± 5.3^{ab} | 45.67 ± 8.66^{ab} | 57.84 ± 11.70^{a} | 33.15 ± 8.09^{bc} | 18.34 ± 2.04^{cd} | 61.62 ± 10.36^{a} | 19.90 ± 2.11^{cd} | 3.64 ± 1.12^{d} |
| testine tumor | | | | | | | | |
| load | | | | | | | | |
| Total Avg. | 35.0 ± 4.5^{ab} | 37.2 ± 6.6^{ab} | 46.6 ± 8.3^{a} | 29.8 ± 5.9^{bc} | 18.5 ± 2.2^{c} | 48.0 ± 7.4^{a} | 18.7 ± 1.9^{c} | 3.4 ± 1.2^{d} |
| tumors/mouse | | | | | | | | |
| Total Avg. | 1.32 ± 0.04^{a} | 1.21 ± 0.03^{bc} | 1.28 ± 0.04^{ab} | 1.20 ± 0.04^{bc} | 1.11 ± 0.04^{cd} | 1.33 ± 0.04^{a} | 1.08 ± 0.02^{d} | 1.04 ± 0.08^{d} |
| overall tumor | | | | | | | | |
| size (mm) | | | | | | | | |
| Total Avg. | 45.91 ± 5.74^{ab} | 45.97 ± 8.88^{ab} | 61.30 ± 12.41^{a} | 35.60 ± 7.10^{bc} | 20.28 ± 2.23^{cd} | 64.84 ± 11.05^{a} | 20.19 ± 2.24^{cd} | 3.64 ± 1.27^{d} |
| overall tumor | | | | | | | | |
| load | | | | | | | | |

Values are expressed as mean ± SEM. Different superscripts indicate values in each row that are significantly different at p < 0.05.
Fisher's least significant difference multiple comparison method is used to determine differences among groups.
Each animal is given equal weight in determining average small intestine tumor size and overall tumor size. Average colon tumor size is calculated only with animals bearing colonic tumors.
Tumor load (=) tumor number × tumor size and is expressed in mm.

As shown in Table 6, the analyses demonstrates that SDA is effective in decreasing polyp number (47%), polyp size (18%) and polyp load (number×size) (56%) in the large intestine and small intestine. It should be noted that while the terms polyp and tumor are used interchangeably, technically speaking, the lesions are polyps (i.e., early stage tumors or neoplasms).

Unexpectedly, the effectiveness of SDA in inhibiting polyp formation and development in the large intestine is comparable to that of sulindac, a NSAID commonly used as a positive control, and is greater than that of not only ALA, but also EPA and DHA. ALA and EPA are marginally efficacious, while DHA shows no efficacy in the large intestine. Likewise, unexpectedly the effectiveness of SDA in inhibiting polyp formation and development in the small intestine is comparable to that of EPA, and is greater than that of ALA and DHA. GLA and CLA, in contrast to SDA, EPA and DHA, appears to increase polyp number; however, the differences are not significant relative to the US17 control.

### EXAMPLE 9

### Effect of n-3 and n-6 PUFAs on Tissue Levels of Arachidonic Acid in the Small Intestine

The efficacy of select n-3 and n-6 polyunsaturated fatty acids in reducing the level of arachidonic acid in small intestine tissue is evaluated using the Min/+ mouse model.

The following fatty acid ethyl esters are tested for their effect on intestinal fatty acid composition: 1) ALA-EE; 2) SDA-EE; 3) EPA-EE; 4) DHA-EE; 5) GLA-EE; and 6) CLA-EE. Fatty acid composition is determined in the small intestine because that is where the vast majority of polyps form.

These fatty acid esters are added to the US17 diet as discussed in Example 8. The arachidonic acid level in the phospholipid fraction of the small intestines of the mice is determined by gas chromatography.

SDA is more effective than ALA, EPA and DHA in decreasing the level of arachidonic acid in the small intestine of the mice. Decreasing the level of arachidonic acid in tissues is desirable because arachidonic acid metabolites have been implicated in tumorigenesis (e.g., prostaglandins, leukotrienes, and HETEs).

### EXAMPLE 10

### Effect of Stearidonic Acid on Primary Tumor Growth in the Nude Mouse/HT-29 Cancer Model

The efficacy of stearidonic acid (18:4n-3) in inhibiting primary tumor growth is evaluated using the nude mouse/HT-29 model. The nude mouse/HT-29 model has been described previously. Hemandez-Alcoceloa R., Fernandez, F., Lacal, J C, Cancer Res., 59(13), 3112-18 (1999); Fantini, J., Cancer J., 5(2) (1992).

Nude (i.e., immunodeficient) mice are fed the US17 diet for three weeks. HT-29 cells are cultured in RPMI-1640 medium supplemented with fetal bovine serum, penicillin, and streptomycin (Gibco, Grand Island, N.Y.) and maintained in a CO₂ atmosphere at 37° C. After achieving the optimal cell density, the HT-29 cells are rinsed and then suspended in phosphate buffered saline (PBS). A cell suspension is made in MATRIGEL (Becton Dickinson Labware, Bedford, Mass.). The suspension is ⅔ by volume cells in PBS and ⅓ by volume MATRIGEL. MATRIGEL provides an extracellular matrix secreted by endothelial cells. The matrix contains angiogenic and cell proliferation growth factors that aid in HT-29 cell attachment and proliferation as a primary tumor.

One million cells are injected in a 30 microliters volume into the subplanter area of the righthind footpad of the nude mice. Five days after the HT-29 cell injections, half of the mice are switched to a US17 diet containing stearidonic acid (3% wt.=10 g/day human equivalent dose) in place of oleic acid. The amount of primary tumor growth is measured by measuring the change in mouse footpad volume over time. Footpad volume is measured with a plethysmometer (Ugo Basile, Camerio-Varese, Italy).

### Rat Carrageenan Foot Pad Edema Test

The carrageenan foot edema test is performed with materials, reagents and procedures essentially as described by Winter, et al., (Proc. Soc. Exp. Biol. Med., 111, 544 (1962)). Male Sprague-Dawley rats are selected in each group so that the average body weight is as close as possible. Rats are fasted with free access to water for over sixteen hours prior to the test. The rats are dosed orally (1 mL) with compounds suspended in vehicle containing 0.5% methylcellulose and 0.025% surfactant, or with vehicle alone. One hour later a subplantar injection of 0.1 mL of 1% solution of carrageenan/sterile 0.9% saline is administered and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Otterness and Bliven, Laboratory Models for Testing NSAIDs, in Non-steroidal Anti-Inflammatory Drugs, (J. Lombardino, ed. 1985)). The % inhibition shows the % decrease from control paw volume determined in this procedure and the data for selected compounds in this invention are summarized in Table 7.

TABLE 7
RAT PAW EDEMA

| | % Inhibition @ |
|---|---|
| Example | 30 mg/kg body weight @ 10 mg/kg body weight |
| 13 | 58 |
| 14 | 65 |
| 25 | 60 |

It is believed that the combinations of the present invention present improvements over the individual components of the combinations.

Other variations and modifications of this invention will be obvious to those skilled in the art. This invention is not limited, except as set forth in the claims.

What is claimed is:

1. A method for treating or preventing a cardiovascular disease in a subject in need of such treatment or prevention, comprising treating the subject with an amount of omega-3 polyunsaturated fatty acid and an amount of a cyclooxygenase-2 selective inhibitor or a pharmaceutically acceptable salt or prodrug thereof wherein the amount of omega-3 polyunsaturated fatty acid and the amount of a cyclooxygenase-2 selective inhibitor or pharmaceutically acceptable salt or prodrug thereof together constitute a cardiovascular disease treatment or prevention effective amount of the omega-3 polyunsaturated fatty acid and the cyclooxygenase-2 selective inhibitor, or a pharmaceutically acceptable salt or prodrug thereof.

2. The method of claim 1 wherein said omega-3 polyunsaturated fatty acid and said cyclooxygenase-2 selective inhibitor or pharmaceutically acceptable salt or prodrug thereof are in a single dosage form.

3. The method of claim 2 wherein said single dosage form comprises about 0.1 to about 10 grams of omega-3 polyunsaturated fatty acid, and about 0.1 to about 2000 milligrams of cyclooxygenase-2 selective inhibitor.

4. The method of claim 1 wherein said cyclooxygenase-2 selective inhibitor is selected from the group consisting of:
8-acetyl-3-(4-fluorophenyl)-2-(4-methylsulfonyl)phenyl-imidazo(1,2-a)pyridine;
5,5-dimethyl-4-(4-methylsulfonyl)phenyl-3-phenyl-2-(5H)-furanone;
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methoxyphenyl)-1H-pyrazol-1-yl)benzenesulfonamide
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5 -(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3 -(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl]spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl]spiro[3.4]oct-6-ene;
5-(3-chloro-4-methoxyphenyl)-6-[4-(methylsulfonyl)phenyl]spiro[2.4]hept-5-ene;
4-[6-(3-chloro-4-methoxyphenyl)spiro[2.4]hept-5-en-5-yl]benzenesulfonamide;
5-(3 ,5 -dichloro-4-methoxyphenyl)-6-[4-(methylsulfonyl)phenyl]spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluoromethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-[(3,5-dichlorophenoxy)methyl)-4-(4-fluorophenyl)-5-[4-(methylsulfonyl)phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene;
4-[4-(4-fluorophenyl)-1,1-dimethylcyclopenta-2,4-dien-3-yl]benzenesulfonamide;
5-(4-fluorophenyl)-6-[4-(methylsulfonyl)phenyl]spiro[2.4]hepta-4,6-diene;
4-[6-(4-fluorophenyl)spiro[2.4]hepta-4,6-dien-5-yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzenesulfonamide;
4-[2-(5-methylpyridin-3-yl)-4-(trifluoromethyl)-1H-imidazol-1-yl]benzenesulfonamide;
4-[2-(2-methylpyridin-3-yl)-4-(trifluoromethyl)-1H-imidazol-1-yl]benzenesulfonamide;
3-[-[4-(methylsulfonyl)phenyl]-4-(trifluoromethyl)-1H-imidazol-2-yl]pyridine;
2-[1-[[4-(methylsulfonyl)phenyl-4-(trifluoromethyl)-1H-imidazol-2-yl]pyridine;
2-methyl-4-[1-[4-(methylsulfonyl)phenyl -4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol-1-yl]benzenesulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl)-1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)-1-[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzenesulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzenesulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1 -ethyl-4-(4-fluorophenyl)-5-(trifluoromethyl)-1H-pyrazol-3-yl]benzenesulfonamide;
N-phenyl-[4-(4-luorophenyl)-3-[4-(methylsulfonyl)phenyl]-5-(trifluoromethyl)-1H-pyrazol-1-yl]acetamide;
ethyl [4-(4-fluorophenyl)-3-[4-(methylsulfonyl)phenyl]-5-(trifluoromethyl)-1H-pyrazol-1-yl]acetate;
4-(4-fluorophenyl)-3-[4-(methylsulfonyl)phenyl]-1-(2-phenylethyl)-1H-pyrazole;
4-(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(methylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl)pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5 -difluoromethyl-3-phenylisoxazol-4-yl]benzenesulfonamide;
4-[5 -hydroxymethyl-3-phenylisoxazol-4-yl]benzenesulfonamide;
4-[5 -methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]-4-(methylsulfonyl)benzene;
4-[2-(4-fluorophenyl)-4,4-dimethylcyclopenten-1-yl]benzenesulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methylsulfonyl)benzene;
1-[2-(2,3-difluorophenyl)cyclopenten-1-yl]-4-(methylsulfonyl)benzene;
4-[2-(3 -fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3-chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl) phenyl]oxazol-2-yl]-2-benzyl-acetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4-(4-fluorophenyl)-2-methyl-5-[4-(methylsulfonyl)phenyl]oxazole; and
4-[5-(3-fluoro-4-methoxyphenyl)-2-trifluoromethyl-4-oxazolyl]benzenesulfonamide.
6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-7-(1,1-dimethylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphthopyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
5,7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
2-trifluoromethyl-3H-naptho[2,1-b]pyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[(phenylmethyl)amino]sulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H-1-benzopyran-3-carboxylic acid;
6-[(4-morpholino)sulfonyl]-2-trifluoromethyl-2H-1-benzopyran-3-carboxylic acid;
6-[(1,-dimethylethy)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(1,1-dimethylethyl)-2-pentafluoroethyl-2H-1-benzopyran-3-carboxylic acid;
5,5-dimethyl-3-(3-fluorophenyl)-4-(4-methyl-sulphonyl-2(5H)-fluranone;
6-chloro-2-trifluoromethyl-2H-1-benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
3-[1-[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4-[2-(5 -methylpyridin-3-yl)-4-(trifluoromethyl)-1H-imidazol-1-yl]benzenesulfonamide;
4-[5-methyl-3-phenylisoxazol-4-yl]benzenesulfonamide;
4-[5 -hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide; or
4-[5-(3-fluoro-4-methoxyphenyl-2-trifluoromethyl)-4-oxazolyl]benzenesulfonamide;
or a pharmaceutically acceptable salt or prodrug thereof.

5. The method according to claim 1, wherein the cyclooxygenase-2 selective inhibitor comprises a compound having the formula: 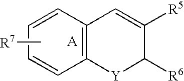
wherein:
Y is selected from the group consisting of O or S or NR^{b};
R^{b} is alkyl;
R⁵ is selected from the group consisting of carboxyl, aminocarbonyl, alkylsulfonylaminocarbonyl and alkoxycarbonyl;
R⁶ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R⁷ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁷ together with ring A forms a naphthyl radical;
or an isomer or prodrug thereof.

6. The method according to claim 5, wherein:
Y is selected from the group consisting of oxygen and sulfur;
R⁵ is selected from the group consisting of carboxyl, lower alkyl, lower aralkyl and lower alkoxycarbonyl;
R⁶ is selected from the group consisting of lower haloalkyl, lower cycloalkyl and phenyl; and
R⁷ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or
wherein R⁷ together with ring A forms a naphthyl radical;
or an isomer or prodrug thereof.

7. The method according to claim 5, wherein:
R⁵ is carboxyl;
R⁶ is lower haloalkyl; and
R⁷ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R⁷ together with ring A forms a naphthyl radical;
or an isomer or prodrug thereof.

8. The method according to claim 5, wherein:
R⁶is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and
R⁷ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R² together with ring A forms a naphthyl radical;
or an isomer or prodrug thereof.

9. The method according to claim 5, wherein:
R⁶ is selected from the group consisting trifluoromethyl and pentafluoroethyl; and
R⁷ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R⁷ together with ring A forms a naphthyl radical;
or an isomer or prodrug thereof.

10. The method of claim 1 wherein said cyclooxygenase-2 selective inhibitor is meloxicam.

11. The method of claim 1 wherein said cyclooxygenase-2 selective inhibitor is 6-[[5-(4-chlorobenzoyl)-1,4-dimethyl-1H-pyrrol-2-yl]methyl]-3 (2H)-pyridazinone, or a pharmaceutically acceptable salt or prodrug thereof.

12. The method of claim 1 wherein said cyclooxygenase-2 selective inhibitor is of the chromene structural class that is a substituted benzopyran or a substituted benzopyran analog, and even more preferably selected from the group consisting of substituted benzothiopyrans, dihydroquinolines, or dihydronaphthalenes having the general Formula II 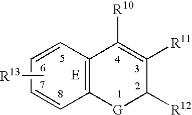
wherein G is selected from the group consisting of O or S or NR^{a}; wherein R^{a} is alkyl;
wherein R¹⁰ is selected from the group consisting of H and aryl
wherein R¹¹ is selected from the group consisting of carboxyl, aminocarbonyl, alkylsulfonylaminocarbonyl and alkoxycarbonyl;
wherein R¹² is selected from the group consisting of haloalkyl, alkyl, aralkyl, cycloalkyl and aryl optionally substituted with one or more radicals selected from alkylthio, nitro and alkylsulfonyl; and
wherein R¹³ is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
or wherein R¹³ together with ring E forms a naphthyl radical;
or a prodrug or isomer or pharmaceutically acceptable salt thereof.

13. The method according to claim 1, wherein the cyclooxygenase-2 selective inhibitor comprises a compound having the formula: 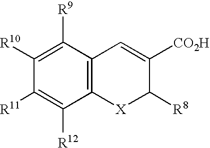
wherein:
X is selected from the group consisting of O and S;
R⁸ is lower haloalkyl;
R⁹ is selected from the group consisting of hydrido, and halo;
R¹⁰ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;
R¹¹ is selected from the group consisting of hydrido, lower alkyl, halo, lower alkoxy, and aryl; and
R¹² is selected from the group consisting of the group consisting of hydrido, halo, lower alkyl, lower alkoxy, and aryl;
or an isomer or prodrug thereof.

14. The method according to claim 13, wherein:
R⁸ is selected from the group consisting of trifluoromethyl and pentafluoroethyl;
R⁹ is selected from the group consisting of hydrido, chloro, and fluoro;
R¹⁰ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;
R¹¹ is selected from the group consisting of hydrido, methyl, ethyl, isopropyl, tert-butyl, chloro, methoxy, diethylamino, and phenyl; and
R¹² is selected from the group consisting of hydrido, chloro, bromo, fluoro, methyl, ethyl, tert-butyl, methoxy, and phenyl;
or an isomer or prodrug thereof.

15. The method of claim 1 wherein said cycloogegenase-2 selective inhibitor is selected from the class of tricyclic cyclooxygenase-2 selective inhibitors represented by the general structure of Formula III 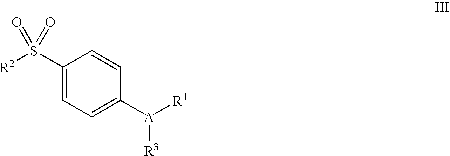
wherein A is selected from the group consisting of partially unsaturated or unsaturated heterocyclyl and partially unsaturated or unsaturated carbocyclic rings;
wherein R¹ is selected from the group consisting of heterocyclyl, cycloalkyl, cycloalkenyl and aryl, wherein R¹ is optionally substituted with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
wherein R² is selected from the group consisting of methyl or amino; and
wherein R³ is a radical selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;
or a pharmaceutically acceptable salt thereof.

16. The method of claim 15 wherein said tricyclic cyclooxygenase-2 inhibitor is celecoxib.

17. The method of claim 15 wherein said tricyclic cyclooxygenase-2 inhibitor is valdecoxib.

18. The method of claim 15 wherein said tricyclic cyclooxygenase-2 inhibitor is deracoxib.

19. The method of claim 15 wherein said tricyclic cyclooxygenase-2 inhibitor is rofecoxib.

20. The method of claim 15 wherein said tricyclic cyclooxygenase-2 inhibitor is etoricoxib, or a pharmaceutically acceptable salt or prodrug thereof.

21. The method of claim 15 wherein said tricyclic cyclooxygenase-2 inhibitor is JTE-522, or a pharmaceutically acceptable salt or prodrug thereof.

22. The method of claim 15 wherein said tricyclic cyclooxygenase-2 inhibitor is parecoxib, or a pharmaceutically acceptable salt thereof.

23. The method according to claim 1, wherein the cyclooxygenase-2 selective inhibitor is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX189, ABT963, meloxicam, prodrugs of any of them, and mixtures thereof.

24. The method according to claim 1, wherein the cyclooxygenase-2 selective inhibitor comprises a phenylacetic acid derivative represented by the general structure: 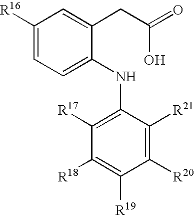
wherein R¹⁶ is methyl or ethyl;
R¹⁷ is chloro or fluoro;
R¹⁸ is hydrogen or fluoro
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not all fluoro when R¹⁶ is ethyl and R¹⁹ is H,
or a prodrug thereof.

25. The method according to claim 24, wherein:
R¹⁶ is ethyl;
R¹⁷ and R¹⁹ are chloro;
R¹⁸ and R²⁰ are hydrogen, and
R²¹ is methyl;
or a prodrug thereof.

26. The method according to claim 1, wherein the cyclooxygenase-2 selective inhibitor comprises a diarylmethylidenefuran derivative.

27. The method according to claim 26, wherein the cyclooxygenase-2 selective inhibitor comprises a diarylmethylidenefuran derivative having the general formula: 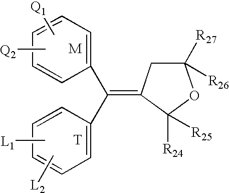
wherein:
the rings T and M independently are:
a phenyl radical,
a naphthyl radical,
a radical derived from a heterocycle comprising 5 to 6 members and possessing from 1 to 4 heteroatoms, or
a radical derived from a saturated hydrocarbon ring having from 3 to 7 carbon atoms;
at least one of the substituents Q₁, Q₂, L₁ or L₂ is:
an —S(O)ₙ—R group, in which n is an integer equal to 0, 1 or 2 and R is a lower alkyl radical having 1 to 6 carbon atoms or a lower haloalkyl radical having 1 to 6 carbon atoms, or an —SO₂NH₂ group;
and is located in the para position,
the others independently being:
a hydrogen atom,
a halogen atom,
a lower alkyl radical having 1 to 6 carbon atoms,
a trifluoromethyl radical, or
a lower O-alkyl radical having 1 to 6 carbon atoms, or
Q₁ and Q₂ or L₁ and L₂ are a methylenedioxy group; and
R₂₄, R₂₅, R₂₆ and R₂₇ independently are:
a hydrogen atom,
a halogen atom,
a lower alkyl radical having 1 to 6 carbon atoms,
a lower haloalkyl radical having 1 to 6 carbon atoms, or
an aromatic radical selected from the group consisting of phenyl, naphthyl, thienyl, furyl and pyridyl; or,
R₂₄, R₂₅ or R₂₆, R₂₇ are an oxygen atom, or
R₂₄, R₂₅ or R₂₆, R₂₇, together with the carbon atom to which they are attached, form a saturated hydrocarbon ring having from 3 to 7 carbon atoms;
or an isomer or prodrug thereof.

28. The method according to claim 27, wherein the cyclooxygenase-2 selective inhibitor comprises a compound selected from the group consisting of N-(2-cyclohexyloxynitrophenyl)methane sulfonamide, and (E)-4-[(4-methylphenyl)(tetrahydro-2-oxo-3-furanylidene) methyl]benzenesulfonamide.

29. The method according to claim 27, wherein the cyclooxygenase-2 selective inhibitor comprises N-(2-cyclohexyloxynitrophenyl)methanesulfonamide.

30. The method according to claim 27, wherein the cyclooxygenase-2 selective inhibitor comprises (E)-4-[(4-methylphenyl)(tetrahydro-2-oxo-3-furanylidene) methyl]benzenesulfonamide.

31. The method according to claim 1, wherein the cyclooxygenase-2 selective inhibitor comprises a material that is selected from the group consisting of nimesulide, flosulide, NS-398, L-745337, RWJ-63556, L-784512, darbufelone, CS-502, LAS-34475, LAS-34555, S-33516, SD-8381, BMS-347070, S-2474, mixtures of any two or more thereof, and prodrugs thereof.

32. The method of claim 1 wherein said omega-3 polyunsaturated fatty acid is α linolenic acid.

33. The method of claim 1 wherein said omega-3 polyunsaturated fatty acid is selected from the group consisting of 9,12,15-octadecatrienoic acid; 6,9,12,15-octadecatetraenoic acid; 8,11,14,17-eicosatetraenoic acid; 5,8,11,14,17-eicosapentaenoic acid; 7,10,13,16,19-docosapentaenoic; and 4,7,10,13,16,19-docosahexaenoic acid.

34. The method of claim 33 wherein said omega-3 polyunsaturated fatty acid is substantially in a cis conformation.

35. The method of claim 33 wherein said omega-3 polyunsaturated fatty acid is 9,12,15-octadecatrienoic acid.

36. The method of claim 33 wherein said omega-3 polyunsaturated fatty acid is 6,9,12,15-octadecatetraenoic acid.

37. The method of claim 33 wherein said omega-3 polyunsaturated fatty acid is 8,11,14,17-eicosatetraenoic acid.

38. The method of claim 33 wherein said omega-3 polyunsaturated fatty acid is 5,8,11,14,17-eicosapentaenoic acid.

39. The method of claim 33 wherein said omega-3 polyunsaturated fatty acid is 7,10,13,16,19 docosapentaenoic.

40. The method of claim 33 wherein said omega-3 polyunsaturated fatty acid is and 4,7,10,13,16,19-docosahexaenoic acid.

41. The method of claim 33 wherein said omega-3 polyunsaturated fatty acid is a combination of α-linolenic acid and Stearidonic acid.

42. The method of claim 33 wherein said omega-3 polyunsaturated fatty acid comprises α-linolenic acid and eicosatetraenoic acid.

43. The method of claim 33 wherein said omega-3 polyunsaturated fatty acid comprises α-linolenic acid and eicosapentaenoic acid.

44. The method of claim 33 wherein said omega-3 polyunsaturated fatty acid comprises α-linolenic acid and docosapentaenoic acid.

45. The method of claim 33 wherein said omega-3 polyunsaturated fatty acid comprises α-linolenic acid and docosahexaenoic acid.

46. The method of claim 33 wherein said omega-3 polyunsaturated fatty acid comprises stearidonic acid and eicosatetraenoic acid.

47. The method of claim 33 wherein said omega-3 polyunsaturated fatty acid comprises stearidonic acid and eicosapentaenoic acid.

48. The method of claim 33 wherein said omega-3 polyunsaturated fatty acid comprises stearidonic acid and docosapentaenoic acid.

49. The method of claim 33 wherein said omega-3 polyunsaturated fatty acid comprises stearidonic acid and docosahexaenoic acid.

50. The method of claim 33 wherein said omega-3 polyunsaturated fatty acid comprises eicosatetraenoic acid, and eicosapentaenoic acid.

51. The method of claim 33 wherein said omega-3 polyunsaturated fatty acid comprises eicosatetraenoic acid, and docosapentaenoic acid.

52. The method of claim 33 wherein said omega-3 polyunsaturated fatty acid comprises eicosatetraenoic acid and docosahexaenoic.

53. The method of claim 33 wherein said omega-3 polyunsaturated fatty acid comprises eicosapentaenoic acid and docosapentaenoic acid.

54. The method of claim 33 wherein said omega-3 polyunsaturated fatty acid comprises eicosapentaenoic acid and docosahexaenoic acid.

55. The method of claim 33 wherein said omega-3 polyunsaturated fatty acid comprises docosapentaenoic acid and docosahexaenoic acid.

56. The method of claim 33 wherein said omega-3 polyunsaturated fatty acid comprises α-linolenic acid, stearidonic acid and eicosatetraenoic acid.

57. The method of claim 33 wherein said omega-3 polyunsaturated fatty acid comprises stearidonic acid, eicosatetraenoic acid and eicosapentaenoic acid.

58. The method of claim 33 wherein said omega-3 polyunsaturated fatty acid comprises eicosatetraenoic acid, eicosapentaenoic acid and docosapentaenoic acid.

59. The method of claim 33 wherein said omega-3 polyunsaturated fatty acid comprises stearidonic acid, docosapentaenoic acid and docosahexaenoic acid.

60. A method of treating or preventing inflammation or inflammatory mediated disease in a subject in need of such treatment or prevention comprising treating the subject with an amount of omega-3 polyunsaturated fatty acid and an amount of a cyclooxygenase-2 selective inhibitor or a pharmaceutically acceptable salt or prodrug thereof wherein the amount of omega-3 polyunsaturated fatty acid and the amount of a cyclooxygenase-2 selective inhibitor or pharmaceutically acceptable salt or prodrug thereof together constitute an inflammation suppressing treatment or prevention effective amount of the omega-3 polyunsaturated fatty acid and the cyclooxygenase-2 selective inhibitor or pharmaceutically acceptable salt or prodrug thereof.

61. The method of claim 60 wherein said omega-3 polyunsaturated fatty acid and said cyclooxygenase-2 selective inhibitor or pharmaceutically acceptable salt or prodrug thereof are in a single dosage form.

62. The method of claim 61 wherein said single dosage form comprises about 0.1 to about 10 grams of omega-3 polyunsaturated fatty acid, and about 0.1 to about 2000 milligrams of cyclooxygenase-2 selective inhibitor.

63. The method of claim 60 wherein said cyclooxygenase-2 selective inhibitor is selected from the group consisting of:
8-acetyl-3-(4-fluorophenyl)-2-(4-methylsulfonyl)phenyl-imidazo(1,2-a)pyridine;
5,5-dimethyl-4-(4-methylsulfonyl)phenyl-3-phenyl-2-(5H)-furanone;
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methoxyphenyl)-1H-pyrazol-1-yl)benzenesulfonamide
4-(3,5-bis(4-methylphenyl)-1H-pyrazol-1-yl)benzenesulfonamide;
4-(5-(4-chlorophenyl)-3-phenyl-1H-pyrazol-1-yl) benzenesulfonamide;
4-(3,5-bis(4-methoxyphenyl)-1H-pyrazol-1-yl)benzenesfonamide;
4-(5-(4-chlorophenyl)-3-(4-methylphenyl)-1H-pyrazol-1-yl]benzenesulfonamide;
4-(5-(4-chlorophenyl)-3-(4-nitrophenyl)-1H -pyrazol-1-yl]benzenesulfonamide;
4-(5-(4-chlorophenyl)-3-(5-chloro-2-thienyl)-1H-pyrazol-1-yl]benzenesulfonamide;
4-(4-chloro-3,5-diphenyl-1H-pyrazol-1-yl)benzenesulfonamide
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl]spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl]spiro[3.4]oct-6-ene;
5-(3-chloro-4-methoxyphenyl)-6-[4-(methylsulfonyl)phenyl]spiro[2.4]hept-5-ene;
4-[6-(3-chloro-4-methoxyphenyl)spiro[2.4]hept-5-en-5-yl]benzenesulfonamide;
5-(3,5-dichloro-4-methoxyphenyl)-6-[4-(methylsulfonyl)phenyl]spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluoromethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-[(3,5-dichlorophenoxy)methyl)-4-(4-fluorophenyl)-5-[4-(methylsulfonyl)phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene;
4-[4-(4-fluorophenyl)-1,1-dimethylcyclopenta-2,4-dien-3-yl]benzenesulfonamide;
5-(4-fluorophenyl)-6-[4-(methylsulfonyl)phenyl]spiro[2.4]hepta-4,6-diene;
4-[6-(4-fluorophenyl)spiro[2.4]hepta-4,6-dien-5 -yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzenesulfonamide;
4-[2-(5-methylpyridin-3-yl)-4-(trifluoromethyl)-1H-imidazol-1-yl]benzenesulfonamide;
4-[2-(2-methylpyridin-3-yl)-4-(trifluoromethyl)-1H-imidazol-1-yl]benzenesulfonamide;
3-[1-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
2-methyl-4-[1-[4-(methylsulfonyl)phenyl-4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol-1-yl]benzenesulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl)-1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)- 1 -[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzenesulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzenesulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzenesulfonamide;
N-phenyl-[4-(4-luorophenyl)-3 -[4-(methylsulfonyl)phenyl]-5-(trifluoromethyl)-1H-pyrazol-1-yl]acetamide;
ethyl [4-(4-fluorophenyl)-3 -[4-(methylsulfonyl)phenyl]-5-(trifluoromethyl)-1H-pyrazol-1-yl]acetate;
4-(4-fluorophenyl)-3-[4-(methylsulfonyl)phenyl]-1-(2-phenylethyl)-1H-pyrazole;
4-(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(methylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl)pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl]benzenesulfonamide;
4-[5-methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]4-(methylsulfonyl)benzene;
4-[2-(4-fluorophenyl)-4,4-dimethylcyclopenten-1-yl]benzene sulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methhlsulfonyl)benzene;
1-[2-(2,3-difluorophenyl)cyclopenten-1-yl]-4-(methylsulfonyl)benzene;
4-[2-(3 -fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3 -chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl) phenyl]oxazol-2-yl]-2-benzyl-acetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4-(4-fluorophenyl)-2-methyl-5-[4-(methylsulfonyl)phenyl]oxazole; and
4-[5-(3-fluoro-4-methoxyphenyl)-2-trifluoromethyl-4-oxazolyl]benzenesulfonamide.
6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-7-(1,1-dimethylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphthopyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluorometyl-2H-1-benzopyran-3-carboxylic acid;
5,7-dichloro-2-trifluorometbyl-2H-1-benzopyran-3-carboxylic acid;
8-phenyl-2-trifluoromethyl-2H-1-benzopyran -3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
2-trifluoromethyl-3H-naptho[2,1-b]pyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1 -benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[(phenylmethyl)amino]sulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H-1-benzopyran-3-carboxylic acid;
6-[(4-morpholino)sulfonyl]-2-trifluoromethyl-2H-1-benzopyran-3-carboxylic acid;
6-[(1,1-dimethylethyl)aminosulfonyl]-2-tri fluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(1,1-dimethylethyl)-2-pentafluoroethyl-2H-1-benzopyran-3-carboxylic acid;
5,5-dimethyl-3-(3-fluorophenyl)-4-(4-methyl-sulphonyl-2(5H)-fluranone;
6-chloro-2-trifluoromethyl-2H-1-benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
3-1-[[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4-[2-(5-methylpyridin-3-yl)-4-(trifluoromethyl)-1H-imidazol-1-yl]benzenesulfonamide;
4-[5-methyl-3-phenylisoxazol-4-yl]benzenesulfonamide;
4-[5-hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide; or
4-[5-(3-fluoro-4-methoxyphenyl-2-trifluoromethyl)-4-oxazolyl]benzenesulfonamide;
or a pharmaceutically acceptable salt or prodrug thereof.

64. The method according to claim 60, wherein the cyclooxygenase-2 selective inhibitor is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX189, ABT963, meloxicam, prodrugs of any of them, and mixtures thereof.

65. The method of claim 60 wherein said cyclooxygenase-2 selective inhibitor is meloxicam.

66. The method of claim 60 wherein said cyclooxygenase-2 selective inhibitor is 6-[[5-(4-chlorobenzoyl)-1,4-dimethyl-1H-pyrrol-2-yl]methyl]-3(2H)-pyridazinone, or a pharmaceutically acceptable salt or prodrug thereof.

67. The method of claim 60 wherein said cyclooxygenase-2 selective inhibitor is of the chromene structural class that is a substituted benzopyran or a substituted benzopyran analog, and even more preferably selected from the group consisting of substituted benzothiopyrans, dihydroquinolines, or dihydronaphthalenes having the general Formula II 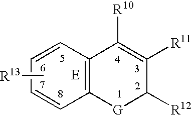
wherein G is selected from the group consisting of O or S or NR^{a}; wherein R^{a} is alkyl;
wherein R¹⁰ is selected from the group consisting of H and aryl
wherein R¹¹ is selected from the group consisting of carboxyl, aminocarbonyl, alkylsulfonylaminocarbonyl and alkoxycarbonyl;
wherein R¹² is selected from the group consisting of haloalkyl, alkyl, aralkyl, cycloalkyl and aryl optionally substituted with one or more radicals selected from alkylthio, nitro and alkylsulfonyl; and
wherein R¹³ is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
or wherein R¹³ together with ring E forms a naphthyl radical;
or a prodrug or isomer or pharmaceutically acceptable salt thereof.

68. The method according to claim 60, wherein the cyclooxygenase-2 selective inhibitor comprises a compound having the formula: 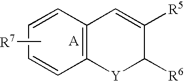
wherein:
Y is selected from the group consisting of O or S or NR^{b};
R^{b} is alkyl;
R⁵ is selected from the group consisting of carboxyl, aminocarbonyl, alkylsulfonylaminocarbonyl and alkoxycarbonyl;
R⁶ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R⁷ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁷ together with ring A forms a naphthyl radical;
or an isomer or prodrug thereof.

69. The method according to claim 68, wherein:
Y is selected from the group consisting of oxygen and sulfur;
R⁵ is selected from the group consisting of carboxyl, lower alkyl, lower aralkyl and lower alkoxycarbonyl;
R⁶ is selected from the group consisting of lower haloalkyl, lower cycloalkyl and phenyl; and
R⁷ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or
wherein R⁷ together with ring A forms a naphthyl radical;
or an isomer or prodrug thereof.

70. The method according to claim 68, wherein:
R⁵ is carboxyl;
R⁶ is lower haloalkyl; and
R⁷ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R⁷ together with ring A forms a naphthyl radical;
or an isomer or prodrug thereof.

71. The method according to claim 68, wherein:
R⁶ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and
R⁷ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R² together with ring A forms a naphthyl radical;
or an isomer or prodrug thereof.

72. The method according to claim 68, wherein:
R⁶ is selected from the group consisting trifluoromethyl and pentafluoroethyl; and
R⁷ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R⁷ together with ring A forms a naphthyl radical;
or an isomer or prodrug thereof.

73. The method according to claim 60, wherein the cyclooxygenase-2 selective inhibitor comprises a compound having the formula: 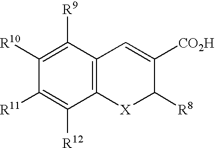
wherein:
X is selected from the group consisting of O and S;
R⁸ is lower haloalkyl;
R⁹ is selected from the group consisting of hydrido, and halo;
R¹⁰ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6- membered nitrogen-containing heterocyclosulfonyl;
R¹¹ is selected from the group consisting of hydrido, lower alkyl, halo, lower alkoxy, and aryl; and
R¹² is selected from the group consisting of the group consisting of hydrido, halo, lower alkyl, lower alkoxy, and aryl;
or an isomer or prodrug thereof.

74. The method according to claim 73, wherein:
R⁸ is selected from the group consisting of trifluoromethyl and pentafluoroethyl;
R⁹ is selected from the group consisting of hydrido, chloro, and fluoro;
R¹⁰ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;
R¹¹ is selected from the group consisting of hydrido, methyl, ethyl, isopropyl, tert-butyl, chloro, methoxy, diethylamino, and phenyl; and
R¹² is selected from the group consisting of hydrido, chloro, bromo, fluoro, methyl, ethyl, tert-butyl, methoxy, and phenyl;
or an isomer or prodrug thereof.

75. The method of claim 60 wherein said cycloogegenase-2 selective inhibitor is selected from the class of tricyclic cyclooxygenase-2 selective inhibitors represented by the general structure of Formula III 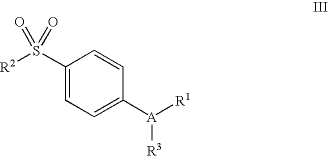
wherein A is selected from the group consisting of partially unsaturated or unsaturated heterocyclyl and partially unsaturated or unsaturated carbocyclic rings;
wherein R¹ is selected from the group consisting of heterocyclyl, cycloalkyl, cycloalkenyl and aryl, wherein R¹ is optionally substituted with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
wherein R² is selected from the group consisting of methyl or amino; and
wherein R³ is a radical selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N- arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;
or a pharmaceutically acceptable salt thereof.

76. The method of claim 75 wherein said tricyclic cyclooxygenase-2 inhibitor is celecoxib.

77. The method of claim 75 wherein said tricyclic cyclooxygenase-2 inhibitor is valdecoxib.

78. The method of claim 75 wherein said tricyclic cyclooxygenase-2 inhibitor is deracoxib.

79. The method of claim 75 wherein said tricyclic cyclooxygenase-2 inhibitor is rofecoxib.

80. The method of claim 75 wherein said tricyclic cyclooxygenase-2 inhibitor is etoricoxib, or a pharmaceutically acceptable salt or prodrug thereof.

81. The method of claim 75 wherein said tricyclic cyclooxygenase-2 inhibitor is JTE-522, or a pharmaceutically acceptable salt or prodrug thereof.

82. The method of claim 75 wherein said tricyclic cyclooxygenase-2 inhibitor is parecoxib, or a pharmaceutically acceptable salt thereof.

83. The method according to claim 60, wherein the cyclooxygenase-2 selective inhibitor comprises a phenylacetic acid derivative represented by the general structure: 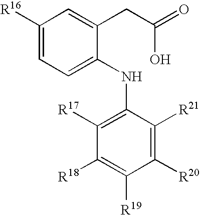
wherein R¹⁶is methyl or ethyl;
R¹⁷ is chloro or fluoro;
R¹⁸ is hydrogen or fluoro
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not allfluoro when R¹⁶ is ethyl and R¹⁹ is H, or a prodrug thereof.

84. The method according to claim 83, wherein:
R¹⁶ is ethyl;
R¹⁷ and R¹⁹ are chloro;
R¹⁸ and R²⁰ are hydrogen, and
R²¹ is methyl;
or a prodrug thereof.

85. The method according to claim 60, wherein the cyclooxygenase-2 selective inhibitor comprises a diarylmethylidenefuran derivative.

86. The method according to claim 85, wherein the cyclooxygenase-2 selective inhibitor comprises a diarylmethylidenefuran derivative having the general formula: 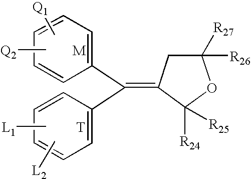
wherein:
the rings T and M independently are:
a phenyl radical,
a naphthyl radical,
a radical derived from a heterocycle comprising 5 to 6 members and possessing from 1 to 4 heteroatoms, or
a radical derived from a saturated hydrocarbon ring having from 3 to 7 carbon atoms;
at least one of the substituents Q₁, Q₂, L₁ or L₂ is:
an —S(O)ₙ—R group, in which n is an integer equal to 0, 1 or 2 and R is a lower alkyl radical having 1 to 6 carbon atoms or a lower haloalkyl radical having 1 to 6 carbon atoms, or an —SO₂NH₂ group;
and is located in the para position,
the others independently being:
a hydrogen atom,
a halogen atom,
a lower alkyl radical having 1 to 6 carbon atoms,
a trifluoromethyl radical, or
a lower O-alkyl radical having 1 to 6 carbon atoms, or
Q₁ and Q₂ or L₁ and L₂ are a methylenedioxy group; and
R₂₄, R₂₅, R₂₆ and R₂₇ independently are:
a hydrogen atom,
a halogen atom,
a lower alkyl radical having 1 to 6 carbon atoms,
a lower haloalkyl radical having 1 to 6 carbon atoms, or
an aromatic radical selected from the group consisting of phenyl, naphthyl, thienyl, furyl and pyridyl; or,
R₂₄, R₂₅ or R₂₆, R₂₇ are an oxygen atom, or
R₂₄, R₂₅ or R₂₆, R₂₇ together with the carbon atom to which they are attached, form a saturated hydrocarbon ring having from 3 to 7 carbon atoms;
or an isomer or prodrug thereof.

87. The method according to claim 86, wherein the cyclooxygenase-2 selective inhibitor comprises a compound selected from the group consisting of N-(2-cyclohexyloxynitrophenyl)methane sulfonamide, and (E)-4-[(4-methylphenyl)(tetrahydro-2-oxo-3-furanylidene) methyl]benzenesulfonamide.

88. The method according to claim 86, wherein the cyclooxygenase-2 selective inhibitor comprises N-(2-cyclohexyloxynitrophenyl)methanesulfonamide.

89. The method according to claim 86, wherein the cyclooxygenase-2 selective inhibitor comprises (E)-4-[(4-methylphenyl)(tetrahydro-2-oxo-3-furanylidene) methyl]benzenesulfonamide.

90. The method according to claim 60, wherein the cyclooxygenase-2 selective inhibitor comprises a material that is selected from the group consisting of nimesulide, flosulide, NS-398, L-745337, RWJ-63556, L-784512, darbufelone, CS-502, LAS-34475, LAS-34555, S-33516, SD-8381, BMS-347070, S-2474, mixtures of any two or more thereof, and prodrugs thereof.

91. The method of claim 60 wherein said omega-3 polyunsaturated fatty acid is o linolenic acid.

92. The method of claim 60 wherein said omega-3 polyunsaturated fatty acid is selected from the group consisting of 9,12,15-octadecatrienoic acid; 6,9,12,15-octadecatetraenoic acid; 8,11,14,17-eicosatetraenoic acid; 5,8,11,14,17-eicosapentaenoic acid; 7,10,13,16,19-docosapentaenoic; and 4,7,10,13,16,19-docosahexaenoic acid.

93. The method of claim 92 wherein said omega-3 polyunsaturated fatty acid is substantially in a cis conformation.

94. The method of claim 92 wherein said omega-3 polyunsaturated fatty acid is 9,12,15-octadecatrienoic acid.

95. The method of claim 92 wherein said omega-3 polyunsaturated fatty acid is 6,9,12,15-octadecatetraenoic acid.

96. The method of claim 92 wherein said omega-3 polyunsaturated fatty acid is 8,11,14,17-eicosatetraenoic acid.

97. The method of claim 92 wherein said omega-3 polyunsaturated fatty acid is 5,8,11,14,17-eicosapentaenoic acid.

98. The method of claim 92 wherein said omega-3 polyunsaturated fatty acid is 7,10,13,16,19 docosapentaenoic.

99. The method of claim 92 wherein said omega-3 polyunsaturated fatty acid is and 4,7,10,13,16,19-docosahexaenoic acid.

100. The method of claim 92 wherein said omega-3 polyunsaturated fatty acid is a combination of α-linolenic acid and Stearidonic acid.

101. The method of claim 92 wherein said omega-3 polyunsaturated fatty acid comprises α-linolenic acid and eicosatetraenoic acid.

102. The method of claim 92 wherein said omega-3 polyunsaturated fatty acid comprises α-linolenic acid and eicosapentaenoic acid.

103. The method of claim 92 wherein said omega-3 polyunsaturated fatty acid comprises α-linolenic acid and docosapentaenoic acid.

104. The method of claim 92 wherein said omega-3 polyunsaturated fatty acid comprises α-linolenic acid and docosahexaenoic acid.

105. The method of claim 92 wherein said omega-3 polyunsaturated fatty acid comprises stearidonic acid and eicosatetraenoic acid.

106. The method of claim 92 wherein said omega-3 polyunsaturated fatty acid comprises stearidonic acid and eicosapentaenoic acid.

107. The method of claim 92 wherein said omega-3 polyunsaturated fatty acid comprises stearidonic acid and docosapentaenoic acid.

108. The method of claim 92 wherein said omega-3 polyunsaturated fatty acid comprises stearidonic acid and docosahexaenoic acid.

109. The method of claim 92 wherein said omega-3 polyunsaturated fatty acid comprises eicosatetraenoic acid, and eicosapentaenoic acid.

110. The method of claim 92 wherein said omega-3 polyunsaturated fatty acid comprises eicosatetraenoic acid, and docosapentaenoic acid.

111. The method of claim 92 wherein said omega-3 polyunsaturated fatty acid comprises eicosatetraenoic acid and docosahexaenoic.

112. The method of claim 92 wherein said omega-3 polyunsaturated fatty acid comprises eicosapentaenoic acid and docosapentaenoic acid.

113. The method of claim 92 wherein said omega-3 polyunsaturated fatty acid comprises eicosapentaenoic acid and docosahexaenoic acid.

114. The method of claim 92 wherein said omega-3 polyunsaturated fatty acid comprises docosapentaenoic acid and docosahexaenoic acid.

115. The method of claim 92 wherein said omega-3 polyunsaturated fatty acid comprises α-linolenic acid, stearidonic acid and eicosatetraenoic acid.

116. The method of claim 92 wherein said omega-3 polyunsaturated fatty acid comprises stearidonic acid, eicosatetraenoic acid and eicosapentaenoic acid.

117. The method of claim 92 wherein said omega-3 polyunsaturated fatty acid comprises eicosatetraenoic acid, eicosapentaenoic acid and docosapentaenoic acid.

118. The method of claim 92 wherein said omega-3 polyunsaturated fatty acid comprises stearidonic acid, docosapentaenoic acid and docosahexaenoic acid.

119. A pharmaceutical composition comprising an omega-3 polyunsaturated fatty acid and a cyclooxygenase-2 selective inhibitor.

120. The pharmaceutical composition of claim 117 wherein said omega-3 polyunsaturated fatty acid is about 0.1 to 10 grams, and said cyclooxygenase-2 selective inhibitor is about 0.1 to 2000 milligrams.

121. The pharmaceutical composition of claim 118 wherein said omega-3 polyunsaturated fatty acid about 0.5 to 7 grams, and said cyclooxygenase-2 selective inhibitor is about 0.5 to 500 milligrams.

122. The pharmaceutical composition of claim 119 wherein said omega-3 polyunsaturated fatty acid about 1 to 5 grams, and said cyclooxygenase-2 selective inhibitor is about 1 to 200 milligrams.

123. A method of treating or preventing cancer or a neoplasia related disorder in a subject in need of such treatment or prevention comprising treating the subject with an amount of omega-3 polyunsaturated fatty acid and an amount of a cyclooxygenase-2 selective inhibitor or a pharmaceutically acceptable salt or prodrug thereof wherein the amount of omega-3 polyunsaturated fatty acid and the amount of a cyclooxygenase-2 selective inhibitor or pharmaceutically acceptable salt or prodrug thereof together constitute an inflammation suppressing treatment or prevention effective amount of the omega-3 polyunsaturated fatty acid and the cyclooxygenase-2 selective inhibitor or pharmaceutically acceptable salt or prodrug thereof.

124. The method of claim 123 wherein wherein the cancer or the neoplasia-related disorder is a malignant tumor growth selected from the group consisting of acral lentiginous melanoma, actinic keratoses, acute lymphocytic leukemia, acute myeloid leukemia, adenocarcinoma, adenoid cycstic carcinoma, adenomas, adenosarcoma, adenosquamous carcinoma, anal canal cancer, anal cancer, anorectum cancer, astrocytic tumors, bartholin gland carcinoma, basal cell carcinoma, biliary cancer, bone cancer, bone marrow cancer, brain cancer, breast cancer, bronchial cancer, bronchial gland carcinomas, carcinoids, carcinoma, carcinosarcoma, cholangiocarcinoma, chondosarcoma, choriod plexus papilloma/carcinoma, chronic lymphocytic leukemia, chronic myeloid leukemia, clear cell carcinoma, colon cancer, colorectal cancer, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, esophageal cancer, Ewing's sarcoma, eye and orbit cancer, female genital cancer, focal nodular hyperplasia, gallbladder cancer, gastric antrum cancer, gastric fundus cancer, gastrinoma, germ cell tumors, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, kidney and renal pelvic cancer, large cell carcinoma, large intestine cancer, larynx cancer, leiomyosarcoma, lentigo maligna melanomas, leukemia, liver cancer, lung cancer, lymphoma, male genital cancer, malignant melanoma, malignant mesothelial tumors, medulloblastoma, medulloepithelioma, melanoma, meningeal cancer, mesothelial cancer, metastatic carcinoma, mouth cancer, mucoepidermoid carcinoma, multiple myeloma, muscle cancer, nasal tract cancer, nervous system cancer, neuroblastoma, neuroepithelial adenocarcinoma nodular melanoma, non-epithelial skin cancer, non-Hodgkin's lymphoma, oat cell carcinoma, oligodendroglial cancer, oral cavity cancer, osteosarcoma, ovarian cancer, pancreatic cancer, papillary serous adenocarcinoma, penile cancer, pharynx cancer, pituitary tumors, plasmacytoma, prostate cancer, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, stomach cancer, striated muscle cancer, submesothelial cancer, superficial spreading melanoma, T cell leukemia, testicular cancer, thyroid cancer, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, and Wilms tumor.

125. The method of claim 123 wherein said omega-3 polyunsaturated fatty acid and said cyclooxygenase-2 selective inhibitor or pharmaceutically acceptable salt or prodrug thereof are in a single dosage form.

126. The method of claim 124 wherein said single dosage form comprises about 0.1 to about 10 grams of omega-3 polyunsaturated fatty acid, and about 0.1 to about 2000 milligrams of cyclooxygenase-2 selective inhibitor.

127. The method of claim 123 wherein said cyclooxygenase-2 selective inhibitor is selected from the group consisting of:
8-acetyl-3-(4-fluorophenyl)-2-(4-methylsulfonyl)phenyl-imidazo(1,2-a)pyridine;
5,5-dimethyl-4-(4-methylsulfonyl)phenyl-3-phenyl-2-(5H)-furanone;
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methoxyphenyl)-1H-pyrazol-1-yl)benzenesulfonamide
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3 ,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5 -(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H- pyrazol-1-yl]benzenesulfonamide;
4-[3-(difluoromethyl)-5-(4-methoxyphenyl)-1H-pyrazol-1-yl]benzenesulfonamide;
4-[3 -cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl]spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl]spiro[3.4]oct-6-ene;
5-(3-chloro-4-methoxyphenyl)-6-[4-(methylsulfonyl)phenyl]spiro[2.4]hept-5-ene;
4-[6-(3-chloro-4-methoxyphenyl)spiro[2.4]hept-5-en-5-yl]benzenesulfonamide;
5-(3 ,5-dichloro-4-methoxyphenyl)-6-[4-(methylsulfonyl)phenyl]spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluoromethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-[(3,5-dichlorophenoxy)methyl)-4-(4-fluorophenyl)-5-[4-(methylsulfonyl)phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene;
4-[4-(4-fluorophenyl)-1,1-dimethylcyclopenta-2,4-dien-3-yl]benzenesulfonamide;
5-(4-fluorophenyl)-6-[4-(methylsulfonyl)phenyl]spiro[2.4]hepta-4,6-diene;
4-[6-(4-fluorophenyl)spiro[2.4]hepta-4,6-dien-5-yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzenesulfonamide;
4-[2-(5-methylpyridin-3-yl)-4-(trifluoromethyl)-1H-imidazol-1-yl]benzenesulfonamide;
4-[2-(2-methylpyridin-3-yl)-4-(trifluoromethyl)-1H-imidazol-1-yl]benzenesulfonamide;
3-[1-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
2-methyl-4-[1-[4-(methylsulfonyl)phenyl-4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol-1-yl]benzenesulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl)-1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)-1-[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzenesulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzenesulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzenesulfonamide;
N-phenyl-[4-(4-luorophenyl)-3-[4-(methylsulfonyl)phenyl]-5-(trifluoromethyl)-1H-pyrazol-1-yl]acetamide;
ethyl [4-(4-fluorophenyl)-3-[4-(methylsulfonyl)phenyl]-5-(trifluoromethyl)-1H-pyrazol-1-yl]acetate;
4-(4-fluorophenyl)-3-[4-(methylsulfonyl)phenyl]-1-(2-phenylethyl)-1H-pyrazole;
4-(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(m ethylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl)pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl]benzenesulfonamide;
4-[5-methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]-4-(methylsulfonyl)benzene;
4-[2-(4-fluorophenyl)-4,4-dimethylcyclopenten-1-yl]benzenesulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methylsulfonyl)benzene;
1-[2-(2,3-difluorophenyl)cyclopenten-1-yl]-4-(methylsulfonyl)benzene;
4-[2-(3-fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3-chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl) phenyl]oxazol-2-yl]-2-benzyl-acetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phen yl]-2-phenyloxazole;
4-(4-fluorophenyl)-2-methyl-5-[4-(methylsulfonyl)phenyl]oxazole; and
4-[5-(3-fluoro-4-methoxyphenyl)-2-trifluoromethyl-4-oxazolyl]benzenesulfonamide.
6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-7-(1,1-dimethylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphthopyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
5,7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro2-trifluoromethyl-2H-1-benzopyran-3-carboxylic acid;
6,8-dichloro-2-trifluoromethyl-2H-1-benzopyran-3-carboxylic acid;
2-trifluoromethyl-3H-naptho[2,1-b]pyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[phenymethyl)amino]sulfonyl]-2-trifluoromethyl-2H-1-benzopyran-3-carboxylic acid;
6-[(dimethylamino)sulfonyl]-2-trifluoromethyl -2H-1-benzopyran-3-carboxylic acid;
6-[( methylamino)sulfonyl]-2-trifluoromethyl-2H- 1-benzopyran-3-carboxylic acid;
6-[(4-morpholino)sulfonyl]-2-trifluoromethyl-2H-1-benzopyran-3-carboxylic acid;
6-[(1,1-dimethylethyl)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(1,1-dimethylethyl)-2-pentafluoroethyl-2H-1-benzopyran-3-carboxylic acid;
5,5-dimethyl-3-(3-fluorophenyl)-4-(4-methyl-sulphonyl-2(5H)-fluranone;
6-chloro-2-trifluoromethyl-2H-1-benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
3-[1-[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4-[2-(5-methylpyridin-3-yl)-4-(trifluoromethyl)-1H-imidazol-1-yl]benzenesulfonamide;
4-[5-methyl-3-phenylisoxazol-4-yl]benzenesulfonamide;
4-[5-hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide; or
4-[5-(3-fluoro-4-methoxyphenyl-2-trifluoromethyl)-4-oxazolyl]benzenesulfonamide;
or a pharmaceutically acceptable salt or prodrug thereof.

128. The method according to claim 123, wherein the cyclooxygenase-2 selective inhibitor is selected from the group consisting of celecoxib, JTE-522, deracoxib, a chromene, a chroman, parecoxib, valdecoxib, etoricoxib, rofecoxib, N-(2-cyclohexyloxynitrophenyl)methane sulfonamide, COX189, ABT963, meloxicam, prodrugs of any of them, and mixtures thereof.

129. The method of claim 123 wherein said cyclooxygenase-2 selective inhibitor is meloxicam.

130. The method of claim 123 wherein said cyclooxygenase-2 selective inhibitor is 6-[[5-(4-chlorobenzoyl)-1,4-dimethyl-1H-pyrrol-2-yl]methyl]-3(2H)-pyridazinone, or a pharmaceutically acceptable salt or prodrug thereof.

131. The method of claim 123 wherein said cyclooxygenase-2 selective inhibitor is of the chromene structural class that is a substituted benzopyran or a substituted benzopyran analog, and even more preferably selected from the group consisting of substituted benzothiopyrans, dihydroquinolines, or dihydronaphthalenes having the general Formula II 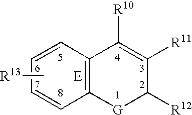
wherein G is selected from the group consisting of O or S or NR^{a}; wherein R^{a} is alkyl;
wherein R¹⁰ is selected from the group consisting of H and aryl
wherein R¹¹ is selected from the group consisting of carboxyl, aminocarbonyl, alkylsulfonylaminocarbonyl and alkoxycarbonyl;
wherein R¹² is selected from the group consisting of haloalkyl, alkyl, aralkyl, cycloalkyl and aryl optionally substituted with one or more radicals selected from alkylthio, nitro and alkylsulfonyl; and
wherein R¹³ is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
or wherein R¹³ together with ring E forms a naphthyl radical;
or a prodrug or isomer or pharmaceutically acceptable salt thereof.

132. The method according to claim 123, wherein the cyclooxygenase-2 selective inhibitor comprises a compound having the formula: 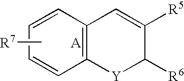
wherein:
Y is selected from the group consisting of O or S or NR^{b};
R^{b} is alkyl;
R⁵ is selected from the group consisting of carboxyl, aminocarbonyl, alkylsulfonylaminocarbonyl and alkoxycarbonyl;
R⁶ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R⁷ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁷ together with ring A forms a naphthyl radical;
or an isomer or prodrug thereof.

133. The method according to claim 132, wherein:
Y is selected from the group consisting of oxygen and sulfur;
R⁵ is selected from the group consisting of carboxyl, lower alkyl, lower aralkyl and lower alkoxycarbonyl;
R⁶ is selected from the group consisting of lower haloalkyl, lower cycloalkyl and phenyl; and
R⁷ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or
wherein R⁷ together with ring A forms a naphthyl radical;
or an isomer or prodrug thereof.

134. The method according to claim 132, wherein:
R⁵ is carboxyl;
R⁶ is lower haloalkyl; and
R⁷ is one or more radicals selected from the group consisting of hydrido, halo, lower alkyl, lower haloalkyl, lower haloalkoxy, lower alkylamino, amino, aminosulfonyl, lower alkylaminosulfonyl, 5 -membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R⁷ together with ring A forms a naphthyl radical;
or an isomer or prodrug thereof.

135. The method according to claim 132, wherein:
R⁶ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and
R⁷ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R² together with ring A forms a naphthyl radical;
or an isomer or prodrig thereof.

136. The method according to claim 132, wherein:
R⁶ is selected from the group consisting trifluoromethyl and pentafluoroethyl; and
R⁷ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R⁷ together with ring A forms a naphthyl radical;
or an isomer or prodrug thereof.

137. The method according to claim 123, wherein the cyclooxygenase-2 selective inhibitor comprises a compound having the formula: 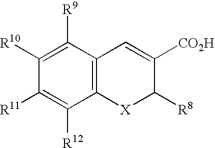
wherein:
X is selected from the group consisting of O and S;
R⁸ is lower haloalkyl;
R⁹ is selected from the group consisting of hydrido, and halo;
R¹⁰ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6- membered nitrogen-containing heterocyclosulfonyl;
R¹¹ is selected from the group consisting of hydrido, lower alkyl, halo, lower alkoxy, and aryl; and
R¹² is selected from the group consisting of the group consisting of hydrido, halo, lower alkyl, lower alkoxy, and aryl;
or an isomer or prodrug thereof.

138. The method according to claim 137, wherein:
R⁸ is selected from the group consisting of trifluoromethyl and pentafluoroethyl;
R⁹ is selected from the group consisting of hydrido, chloro, and fluoro;
R¹⁰ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;
R¹¹ is selected from the group consisting of hydrido, methyl, ethyl, isopropyl, tert-butyl, chloro, methoxy, diethylamino, and phenyl; and
R¹² is selected from the group consisting of hydrido, chloro, bromo, fluoro, methyl, ethyl, tert-butyl, methoxy, and phenyl;
or an isomer or prodrug thereof.

139. The method of claim 123 wherein said cycloogegenase-2 selective inhibitor is selected from the class of tricyclic cyclooxygenase-2 selective inhibitors represented by the general structure of Formula III 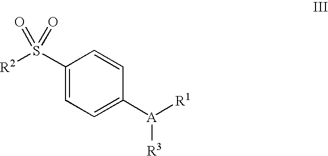
wherein A is selected from the group consisting of partially unsaturated or unsaturated heterocyclyl and partially unsaturated or unsaturated carbocyclic rings;
wherein R¹ is selected from the group consisting of heterocyclyl, cycloalkyl, cycloalkenyl and aryl, wherein R¹ is optionally substituted with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
wherein R² is selected from the group consisting of methyl or amino; and
wherein R³ is a radical selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N- arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;
or a pharmaceutically acceptable salt thereof.

140. The method of claim 139 wherein said tricyclic cyclooxygenase-2 inhibitor is celecoxib.

141. The method of claim 139 wherein said tricyclic cyclooxygenase-2 inhibitor is valdecoxib.

142. The method of claim 139 wherein said tricyclic cyclooxygenase-2 inhibitor is deracoxib.

143. The method of claim 139 wherein said tricyclic cyclooxygenase-2 inhibitor is rofecoxib.

144. The method of claim 139 wherein said tricyclic cyclooxygenase-2 inhibitor is etoricoxib, or a pharmaceutically acceptable salt or prodrug thereof.

145. The method of claim 139 wherein said tricyclic cyclooxygenase-2 inhibitor is JTE-522, or a pharmaceutically acceptable salt or prodrug thereof.

146. The method of claim 139 wherein said tricyclic cyclooxygenase-2 inhibitor is parecoxib, or a pharmaceutically acceptable salt thereof.

147. The method according to claim 123, wherein the cyclooxygenase-2 selective inhibitor comprises a phenylacetic acid derivative represented by the general structure: 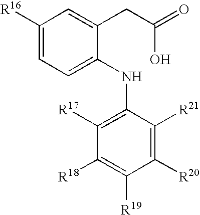
wherein R¹⁶ is methyl or ethyl;
R¹⁷ is chloro or fluoro;
R¹⁸ is hydrogen or fluoro
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not all fluoro when R¹⁶ is ethyl and R¹⁹ is H, or a prodrug thereof.

148. The method according to claim 147, wherein:
R¹⁶ is ethyl;
R¹⁷ and R¹⁹ are chloro;
R¹⁸ and R²⁰ are hydrogen, and
R²¹ is methyl;
or a prodrug thereof.

149. The method according to claim 123, wherein the cyclooxygenase-2 selective inhibitor comprises a diarylmethylidenefuran derivative.

150. The method according to claim 149, wherein the cyclooxygenase-2 selective inhibitor comprises a diarylmethylidenefuran derivative having the general formula: 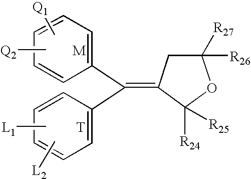
wherein:
the rings T and M ind ndently are:
a phenyl radical,
a naphthyl radical,
a radical derived from a heterocycle comprising 5 to 6 members and possessing from 1 to 4 heteroatoms, or
a radical derived from a saturated hydrocarbon ring having from 3 to 7 carbon atoms;
at least one of the substituents Q₁, Q₂, L₁ or L₂ is:
an —S(O)ₙ—R group, in which n is an integer equal to 0, 1 or 2 and R is a lower alkyl radical having 1 to 6 carbon atoms or a lower haloalkyl radical having 1 to 6 carbon atoms, or an —SO₂NH₂ group;
and is located in the para position,
the others independently being:
a hydrogen atom,
a halogen atom,
a lower alkyl radical having 1 to 6 carbon atoms,
a trifluoromethyl radical, or
a lower O-alkyl radical having 1 to 6 carbon atoms, or
Q₁ and Q₂ or L₁ and L₂ are a methylenedioxy group; and
R₂₄, R₂₅, R₂₆ and R₂₇ independently are:
a hydrogen atom,
a halogen atom,
a lower alkyl radical having 1 to 6 carbon atoms,
a lower haloalkyl radical having 1 to 6 carbon atoms, or
an aromatic radical selected from the group consisting of phenyl, naphthyl, thienyl, furyl and pyridyl; or,
R₂₄, R₂₅ or R₂₆, R₂₇ are an oxygen atom, or
R₂₄, R₂₅ or R₂₆, R₂₇, together with the carbon atom to which they are attached, form a saturated hydrocarbon ring having from 3 to 7 carbon atoms;
or an isomer or prodrug thereof.

151. The method according to claim 150, wherein the cyclooxygenase-2 selective inhibitor comprises a compound selected from the group consisting of N-(2-cyclohexyloxynitrophenyl)methane sulfonamide, and (E)-4-[(4-methylphenyl)(tetrahydro-2-oxo-3-furanylidene) methyl]benzenesulfonamide.

152. The method according to claim 150, wherein the cyclooxygenase-2 selective inhibitor comprises N-(2-cyclohexyloxynitrophenyl)methanesulfonamide.

153. The method according to claim 150, wherein the cyclooxygenase-2 selective inhibitor comprises (E)-4-[(4-methylphenyl)(tetrahydro-2-oxo-3-furanylidene) methyl]benzenesulfonamide.

154. The method according to claim 123, wherein the cyclooxygenase-2 selective inhibitor comprises a material that is selected from the group consisting of nimesulide, flosulide, NS-398, L-745337, RWJ-63556, L-784512, darbufelone, CS-502, LAS-34475, LAS-34555, S-33516, SD-8381, BMS-347070, S-2474, mixtures of any two or more thereof, and prodrugs thereof.

155. The method of claim 123 wherein said omega-3 polyunsaturated fatty acid is α linolenic acid.

156. The method of claim 123 wherein said omega-3 polyunsaturated fatty acid is selected from the group consisting of 9,12,15-octadecatrienoic acid; 6,9,12,15-octadecatetraenoic acid; 8,11,14,17-eicosatetraenoic acid; 5,8,11,14,17-eicosapentaenoic acid; 7,10,13,16,19-docosapentaenoic; and 4,7,10,13,16,19-docosahexaenoic acid.

157. The method of claim 156 wherein said omega-3 polyunsaturated fatty acid is substantially in a cis conformation.

158. The method of claim 156 wherein said omega-3 polyunsaturated fatty acid is 9,12,15-octadecatrienoic acid.

159. The method of claim 156 wherein said omega-3 polyunsaturated fatty acid is 6,9,12,15-octadecatetraenoic acid.

160. The method of claim 156 wherein said omega-3 polyunsaturated fatty acid is 8,11,14,17-eicosatetraenoic acid.

161. The method of claim 156 wherein said omega-3 polyunsaturated fatty acid is 5,8,11,14,17-eicosapentaenoic acid.

162. The method of claim 156 wherein said omega-3 polyunsaturated fatty acid is 7,10,13,16,19 docosapentaenoic.

163. The method of claim 156 wherein said omega-3 polyunsaturated fatty acid is and 4,7,10,13,16,19-docosahexaenoic acid.

164. The method of claim 156 wherein said omega-3 polyunsaturated fatty acid is a combination of α-linolenic acid and Stearidonic acid.

165. The method of claim 156 wherein said omega-3 polyunsaturated fatty acid comprises α-linolenic acid and eicosatetraenoic acid.

166. The method of claim 156 wherein said omega-3 polyunsaturated fatty acid comprises α-linolenic acid and eicosapentaenoic acid.

167. The method of claim 156 wherein said omega-3 polyunsaturated fatty acid comprises α-linolenic acid and docosapentaenoic acid.

168. The method of claim 156 wherein said omega-3 polyunsaturated fatty acid comprises α-linolenic acid and docosahexaenoic acid.

169. The method of claim 156 wherein said omega-3 polyunsaturated fatty acid comprises stearidonic acid and eicosatetraenoic acid.

170. The method of claim 156 wherein said omega-3 polyunsaturated fatty acid comprises stearidonic acid and eicosapentaenoic acid.

171. The method of claim 156 wherein said omega-3 polyunsaturated fatty acid comprises stearidonic acid and docosapentaenoic acid.

172. The method of claim 156 wherein said omega-3 polyunsaturated fatty acid comprises stearidonic acid and docosahexaenoic acid.

173. The method of claim 156 wherein said omega-3 polyunsaturated fatty acid comprises eicosatetraenoic acid, and eicosapentaenoic acid.

174. The method of claim 156 wherein said omega-3 polyunsaturated fatty acid comprises eicosatetraenoic acid, and docosapentaenoic acid.

175. The method of claim 156 wherein said omega-3 polyunsaturated fatty acid comprises eicosatetraenoic acid and docosahexaenoic.

176. The method of claim 156 wherein said omega-3 polyunsaturated fatty acid comprises eicosapentaenoic acid and docosapentaenoic acid.

177. The method of claim 156 wherein said omega-3 polyunsaturated fatty acid comprises eicosapentaenoic acid and docosahexaenoic acid.

178. The method of claim 156 wherein said omega-3 polyunsaturated fatty acid comprises docosapentaenoic acid and docosahexaenoic acid.

179. The method of claim 156 wherein said omega-3 polyunsaturated fatty acid comprises α-linoleic acid, stearidonic acid and eicosatetraenoic acid.

180. The method of claim 156 wherein said omega-3 polyunsaturated fatty acid comprises stearidonic acid, eicosatetraenoic acid and eicosapentaenoic acid.

181. The method of claim 156 wherein said omega-3 polyunsaturated fatty acid comprises eicosatetraenoic acid, eicosapentaenoic acid and docosapentaenoic acid.

182. The method of claim 156 wherein said omega-3 polyunsaturated fatty acid comprises stearidonic acid, docosapentaenoic acid and docosahexaenoic acid.
